(19) United States
(12) Patent Application Publication
(10) Pub. No.: US 2023/0218773 A1
(43) Pub. Date: Jul. 13, 2023

(54) ALBUMIN DRUG CONJUGATES AND USE THEREOF FOR THE TREATMENT OF CANCER

(71) Applicant: BOARD OF REGENTS, THE UNIVERSITY OF TEXAS SYSTEM, Austin, TX (US)

(72) Inventors: Debadyuti GHOSH, Austin, TX (US); Xinquan LIU, Austin, TX (US)

(73) Assignee: BOARD OF REGENTS, THE UNIVERSITY OF TEXAS SYSTEM, Austin, TX (US)

(21) Appl. No.: 17/997,453

(22) PCT Filed: Apr. 30, 2021
(86) PCT No.: PCT/US2021/030187

§ 371 Date: Oct. 28, 2022

Related U.S. Application Data
(60) Provisional application No. 63/018,233, filed on Apr. 30, 2020.

(51) Int. Cl. A61K 47/64; A61K 47/65; A61P 35/00; A61P 1/18; A61K 45/06

(52) U.S. Cl. CPC A61K 47/643; A61K 47/65; A61P 35/00; A61P 1/18; A61K 45/06

(57) ABSTRACT

Provided herein are methods for producing an albumin drug conjugate. The albumin and dmg may be mixed ex vivo prior to administration. Further provided herein are methods of treating cancer comprising administering the albumin drug conjugate.

This application claims benefit of priority to U.S. Provisional Application Ser. No. 63/018,233, filed Apr. 30, 2020, the entire contents of which are hereby incorporated by reference.

### BACKGROUND

### 1. Field

The present invention relates generally to the field of molecular biology. More particularly, it concerns methods of albumin drug conjugates and methods of use thereof.

### 2. Description of Related Art

Chemotherapy drugs have been widely used as standard of care to treat cancers for several decades.[1] However, due to their promiscuous cytotoxicity, they can cause adverse events and have narrow dosing regimens, which limit their efficacy. In part to address these challenges, drug delivery systems have been developed to improve the therapeutic index of existing chemotherapy drugs, including drug derivates, micelles, liposomes, and polymeric nanoparticles. [2, 3] In principle, these delivery systems can extend the pharmacokinetics of the free drug and/or increase the accumulation of drug in tumor sites, thereby minimizing the toxic side effects caused by non-specific distribution of chemotherapies.

In particular, albumin is attractive as a natural drug delivery system. It is the most abundant protein in plasma, has a long circulation half-life (˜19 days), and has been shown to improve the pharmacokinetic profiles of payloads.[4, 5] For example, therapeutic peptides and proteins have been attached to myristic acid, which has a high binding affinity to endogenous albumin, to improve their half-life from several minutes to about 10 hours. Leveraging the intrinsic pharmacokinetics of albumin led to the development of Levemir® and Victoza® to treat diabetes.[6] In cancer, albumin has been shown to accumulate in tumors and thus modify the distribution of the payloads.[7] Nanoparticle albumin bound paclitaxel (nab-paclitaxel) in combination with gemcitabine exhibited improved overall survival compared to gemcitabine monotherapy in patients with advanced pancreatic cancer and is now standard of care. [8]

In addition to these strategies, the naturally available thiol at position cysteine 34 (Cys34) has been exploited for site-specific conjugation of drugs, prodrugs, and polymers.[9-12] Cys34 is located in a shallow crevice between two α-helices in subdomain IA and is amenable for covalent conjugation. Previously, several chemotherapy drugs have been synthesized to bind Cys34 through an acid sensitive or enzyme cleavable linker with a maleimide group. These drug-linkers were designed to bind to endogenous albumin after administration to form in situ albumin-drug conjugates.[13-16] The most advanced prodrug based on this design strategy is an albumin-binding prodrug of doxorubicin (aldoxorubicin), a maleimide activated prodrug with an acid-sensitive hydrazone linker, which has completed evaluation in a Phase III clinical trial for relapsed or refractory soft-tissue sarcoma.[17] Aldoxorubicin was administered safely with a 3.47-fold higher dose compared to doxorubicin with minimal cardiac toxicity. The improved response rate and progression free survival in Phase II and III trials were statistically significant over doxorubicin.[18-20] However, in a Phase II clinical trial with previously untreated soft-tissue sarcoma, the median overall survival was not considerably different between aldoxorubicin and doxorubicin arms (15.8% vs. 14.3%). [20]

While prodrugs that bind to endogenous albumin are promising, there remain challenges that need to be resolved to fully realize the potential of albumin-based carriers for drug delivery. In situ binding of prodrugs to circulating albumin through Cys34 is not specific because the maleimide group on these prodrugs can also react with other cysteines and lysines present on other blood proteins and cells.[21-23] The resulting non-specific binding can increase off-target uptake and reduce the delivered dose in target tissue, thereby impairing drug efficacy in vivo. There is evidence that doxorubicin conjugated to an albumin-binding peptide, which binds to endogenous albumin, outperformed the albumin-binding prodrug by improving the binding specificity.[23] As a result, it is necessary to develop alternative approaches that can improve the therapeutic index of highly potent chemotherapeutic agents.

### SUMMARY

In certain embodiments, the present disclosure provides a composition comprising an albumin drug conjugate comprising recombinant human albumin. In some aspects, the drug is conjugated to Cysteine 34 of albumin. In certain aspects, the drug and recombinant human albumin are conjugated ex vivo.

In some aspects, the albumin is recombinant human serum albumin. In particular aspects, the drug conjugate is free of endogenous albumin. In certain aspects, recombinant human serum albumin is at a concentration of 5 mg/mL to 15 mg/mL, such as about 5 ng/mL, 6 ng/mL, 7 ng/mL, 8 g/mL, 9 g/mL, 10 g/mL, 11 g/mL, 12 g/mL, 13 g/mL, 14 g/mL, or 15 g/mL. In particular aspects, the recombinant human serum albumin is at a concentration of about 10 mg/mL.

In some aspects, the composition further comprises a linker between the drug and albumin. In certain aspects, the linker is a cleavable linker. In particular aspects, the linker conjugates to a free thiol of Cysteine 34 of albumin. In some aspects, the linker is an enzyme sensitive linker (e.g., protease sensitive linker), a pH-sensitive linker (e.g., hydrozone linker), or a reducible linker (disulfide bond linker). In specific aspects, the protease is cathepsin (e.g., cathepsin-B, cathepsin S, cathepsin L, cathepsin, cathepsin D, cathepsin E, or cathepsin K), matrix metalloproteinase (e.g., MMP1, MMP2, MMP3, MMP7, MMP8, MMP9, MMP12, or MMP14), caspase-3, A disintegrin and metalloproteinase (ADAM) (e.g., ADAM10 or ADAM17), kallekrin-related peptidase (e.g., KLK1, KLK2, KLK3, KLK6, or KLK7), urokinase plasminogen activator (uPA), hepsin (HPN), matripase, legumain, dipeptidyl peptidase (DPP4), or fibroblast activation protein (FAP). In particular aspects, the protease is cathepsin-B. In some aspects, the cleavable linker is a valine-citrulline dipeptide linker, such as a cathepsin-B sensitive valine-citrulline dipeptide linker.

In particular aspects, the albumin is and drug-linker conjugate are at a molar ratio of 1:1 to 1:5. In some aspects, the albumin and a drug-linker conjugate are at a molar ratio of 1:3.

In further aspects, the albumin drug conjugate further comprises a spacer. In some aspects, the spacer is a p-aminobenzyl carbamate (PABC) spacer. In some aspects, the spacer is a PEG spacer (e.g., Mal-dPEG4-NHS) or carbamoyl sulfamide linker. In particular aspects, the spacer is located between the drug the the linker.

In some aspects, the molar ratio of drug to albumin is 1:1 to 3:1. In particular aspects, the molar ratio of drug to albumin is 1:1.

In some aspects, the drug is an anti-cancer agent. In certain aspects, the drug is a chemotherapeutic, radiotherapeutic, gene therapy, hormonal therapy, anti-angiogenic therapy or immunotherapy. In particular aspects, the anti-cancer agent is a SHP inhibitor (e.g., SHP099), a SOS inhibitor (e.g., BAY293 or BI3406), a maytansinoid, an auristatin (e.g., MMAE or MMAF), calicheamicin, an anthracycline, a taxane, a MEK inhibitor (e.g., selumetinib, trametinib. cobimetinib, and binimetinib), a poly (adenosine diphosphate ribose) polymerase (PARP) inhibitor, a RAF inhibitor (e.g., vemurafenib and dabrafenib), a KRAS G12C inhibitor (e.g., cysteine-reactive, covalent KRAS G12C inhibitors including AMG 510 (NCT03600883 or NCT04185883), MRTX-849 (NCT03785249), JNJ-74699157 (NCT03114319), or LY3499446 (NCT04165031)), platinum-based compound, anthracycline, or topoisomerase I inhibitor. In specific aspects, the anti-cancer agent is a chemotherapeutic agent. In particular aspects, the drug is monomethyl auristatin E (MMAE) or gemcitabine. In some aspects, the chemotherapeutic agent is anthracycline, camptothecin, paclitaxel, auristatin, or docetaxel.

A further embodiment provides a method for producing an albumin drug conjugate comprising covalently conjugating a drug to Cysteine 34 of albumin, wherein the conjugation is performed ex vivo.

In some aspects, the albumin is recombinant human serum albumin. In particular aspects, the drug conjugate is free of endogenous albumin. In certain aspects, recombinant human serum albumin is at a concentration of 5 mg/mL to 15 mg/mL, such as about 5 ng/mL, 6 ng/mL, 7 ng/mL, 8 g/mL, 9 g/mL, 10 g/mL, 11 g/mL, 12 g/mL, 13 g/mL, 14 g/mL, or 15 g/mL. In particular aspects, the recombinant human serum albumin is at a concentration of about 10 mg/mL.

In some aspects, the drug is conjugated to a linker prior to conjugating to albumin. In certain aspects, the linker is a cleavable linker. In particular aspects, the linker conjugates to a free thiol of Cysteine 34 of albumin. In some aspects, the linker is an enzyme sensitive linker (e.g., protease sensitive linker), a pH-sensitive linker (e.g., hydrozone linker), or a reducible linker (disulfide bond linker). In specific aspects, the protease is cathepsin (e.g., cathepsin-B, cathepsin S, cathepsin L, cathepsin, cathepsin D, cathepsin E, or cathepsin K), matrix metalloproteinase (e.g., MMP1, MMP2, MMP3, MMP7, MMP8, MMP9, MMP12, or MMP14), caspase-3, A disintegrin and metalloproteinase (ADAM) (e.g., ADAM10 or ADAM17), kallekrin-related peptidase (e.g., KLK1, KLK2, KLK3, KLK6, or KLK7), urokinase plasminogen activator (uPA), hepsin (HPN), matripase, legumain, dipeptidyl peptidase (DPP4), or fibroblast activation protein (FAP). In particular aspects, the protease is cathepsin-B. In some aspects, the cleavable linker is a valine-citrulline dipeptide linker, such as a cathepsin-B sensitive valine-citrulline dipeptide linker.

In particular aspects, the albumin is added to a drug-linker conjugate at a molar ratio of 1:1 to 1:5. In some aspects, the albumin is added to a drug-linker conjugate at a molar ratio of 1:3. In certain aspects, excess drug-linker conjugate is removed by a desalting column or flow filtration. In some aspects, the albumin is dissolved in phosphate buffered saline. In certain aspects, the drug-linker conjugate is dissolved in acetonitrile.

In further aspects, the albumin drug conjugate further comprises a spacer. In some aspects, the spacer is a p-aminobenzyl carbamate (PABC) spacer. In some aspects, the spacer is a PEG spacer (e.g., Mal-dPEG4-NHS) or carbamoyl sulfamide linker. In particular aspects, the spacer is located between the drug the the linker.

In some aspects, the molar ratio of drug to albumin is 1:1 to 3:1. In particular aspects, the molar ratio of drug to albumin is 1:1.

In certain aspects, the method further comprises reducing albumin to expose reactive thiols prior to conjugation. In some aspects, reducing comprises the addition of tris(2-carboxyethyl) phosphine hydrochloride (TCEP).

In some aspects, the drug is an anti-cancer agent. In certain aspects, the drug is a chemotherapeutic, radiotherapeutic, gene therapy, hormonal therapy, anti-angiogenic therapy or immunotherapy. In particular aspects, the anti-cancer agent is a SHP inhibitor (e.g., SHP099), a SOS inhibitor (e.g., BAY293 or BI3406), a maytansinoid, an auristatin (e.g., MMAE or MMAF), calicheamicin, an anthracycline, a taxane, a MEK inhibitor (e.g., selumetinib, trametinib. cobimetinib, and binimetinib), a poly (adenosine diphosphate ribose) polymerase (PARP) inhibitor, a RAF inhibitor (e.g., vemurafenib and dabrafenib), a KRAS G12C inhibitor (e.g., cysteine-reactive, covalent KRAS G12C inhibitors including AMG 510 (NCT03600883 or NCT04185883), MRTX-849 (NCT03785249), JNJ-74699157 (NCT03114319), or LY3499446 (NCT04165031)), platinum-based compound, anthracycline, or topoisomerase I inhibitor. In specific aspects, the anti-cancer agent is a chemotherapeutic agent. In particular aspects, the drug is monomethyl auristatin E (MMAE) or gemcitabine. In some aspects, the chemotherapeutic agent is anthracycline, camptothecin, paclitaxel, auristatin, or docetaxel.

Another embodiment provides a pharmaceutical composition comprising an albumin drug conjugate wherein the albumin is recombinant human serum albumin. The human serum albumin may be produced in bacterial, yeast, or mammalian cells. In some aspects, the albumin is conjugated to the drug by a cleavable linker. In certain aspects, the linker is a cleavable linker. In particular aspects, the linker conjugates to a free thiol of Cysteine 34 of albumin. In some aspects, the linker is a protease sensitive linker, a pH-sensitive linker (e.g., hydrozone linker), or a reducible linker (disulfibond linker). In specific aspects, the protease is cathepsin (e.g., cathepsin-B, cathepsin S, cathepsin L, cathepsin, cathepsin D, cathepsin E, or cathepsin K), matrix metalloproteinase (e.g., MMP1, MMP2, MMP3, MMP7, MMP8, MMP9, MMP12, or MMP14), caspase-3, A disintegrin and metalloproteinase (ADAM) (e.g., ADAM10 or ADAM17), kallekrin-related peptidase (e.g., KLK1, KLK2, KLK3, KLK6, or KLK7), urokinase plasminogen activator (uPA), hepsin (HPN), matripase, legumain, dipeptidyl peptidase (DPP4), or fibroblast activation protein (FAP). In particular aspects, the protease is cathepsin-B. In some aspects, the cleavable linker is a valine-citrulline dipeptide linker, such as a cathepsin-B sensitive valine-citrulline dipeptide linker. In some aspects, the conjugate is produced according to the method of the present embodiments or aspects thereof.

A further embodiment provides a method of delivering a drug into a tumor cell comprising administering an effective amount of an albumin drug conjugate of the present embodiments or aspects thereof to said cell.

In another embodiment, there is provided the use of an albumin drug conjugate of the present embodiments and aspects thereof for the treatment of cancer in a subject.

In some aspects, the cancer is a RAS mutant cancer. In certain aspects, the RAS mutant cancer is pancreatic cancer (e.g., pancreatic ductal adenocarcinoma), colorectal cancer (e.g., colorectal adenocarcinoma), or lung cancer (e.g., non-small cell lung cancer). In certain aspects, the cancer is pancreatic cancer. In some aspects, the subject is human.

Another embodiment provides a method of treating cancer in a subject comprising administering an effective amount of an albumin drug conjugate of any of the present embodiments or aspects thereof to said subject. In some aspects, the cancer is a RAS mutant cancer. In certain aspects, the RAS mutant cancer is pancreatic cancer (e.g., pancreatic ductal adenocarcinoma), colorectal cancer (e.g., colorectal adenocarcinoma), or lung cancer (e.g., non-small cell lung cancer). In particular aspects, the cancer is pancreatic cancer.

In some aspects, the subject is a human. In certain aspects, the albumin drug conjugate is administered orally, topically, intravenously, intraperitoneally, intramuscularly, endoscopically, percutaneously, subcutaneously, regionally, or by direct injection. In particular aspects, the albumin drug conjugate is administered intravenously.

In additional aspects, the method further comprises administering at least a second therapeutic agent. In some aspects, the at least a second therapeutic agent is an anti-cancer agent. In certain aspects, the at least a second therapeutic is chemotherapy, radiotherapy, gene therapy, surgery, hormonal therapy, anti-angiogenic therapy or immunotherapy (e.g., an immune checkpoint inhibitor, such as is an anti-PD1 antibody or anti-CTLA-4 antibody, or a cytokine, such as IL-2 or IL-12). In some aspects, the immune checkpoint inhibitor is an inhibitor of an inhibitor of CTLA-4, PD-1, PD-L1, PD-L2, LAG-3, BTLA, B7H3, B7H4, TIM3, KIR, or A2aR, TIGIT, or VISTA. In some aspects, the second therapeutic agent is a STING agonist. In some aspects, the albumin drug conjugate has improved half-life, anti-tumor efficacy, and/or is delivered at a higher dose to a tumor as compared to an albumin drug conjugated in vivo.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: (A) Structure of albumin: cysteine 34, disulfide bonds. (B) Scheme of syntheses of albumin-drug conjugates. (C) Schematic of albumin-drug conjugate administration.

FIGS. 2A-2C: Molecular weights of rHSA (A), ALDC1 (B) and ALDC3 (C) determined by LC-MS.

FIG. 3: MMAE released from albumin-drug conjugates in buffer (pH 5.5) with/without Cathepsin B at 37° C.

FIG. 4: Far-UV circular dichroism spectra of albumin and albumin-drug conjugates.

FIGS. 5A-5D: (A) Calculated IC50 of MMAE on human pancreatic cancer cells MIA PaCa2 and PANC1 and human umbilical vein endothelial cells (HUVEC). IC50 of ALDC1 and ALDC3 on (B) MIA PaCa2 cells, (C) PANC1 cells, and (D) HUVEC cells). Data shown as mean±standard deviation (n=3).

FIGS. 6A-6B: Plasma concentration of (A) Free MMAE and (B) Total MMAE after a single intravenous injection of MMAE, MMAE-MAL, ALDC1 and ALDC3 containing 0.5 mg/kg MMAE. Data shown as mean±SEM (n=3). Note that the initial time point is 10 minutes (0.167 hours) and not 0 minutes.

FIGS. 7A-7D: Tissue distribution of free MMAE (A) and total MMAE (B) in tumor, live and kidney 24 hours after a single intravenous injection containing 0.5 mg/kg MMAE. Concentrations (unit: ng per gram tissue) of free MMAE (C) and total MMAE (D) determined in tumor. Data shown as mean±SEM. (**p<0.01, ***p<0.001, ****p<0.0001, ns means no significant difference)

FIGS. 8A-8C: (A) Antitumor efficacy of albumin drug conjugates in MIA PaCa2 tumor-bearing mice. Treatment started when tumor volume was about 150 mm³. MMAE, albumin-drug conjugates or control were dosed at Day 0, 4, 8, and 12. Vehicle was rHSA and dosed at 36 mg/kg. MMAE, MMAE-MAL, ALDC1 and ALDC3 were dosed at 0.5 mg/kg. ALDC1-H was dosed at 0.9 mg/kg. (n=6, Data were presented as mean+SEM). (B) Prolonged survival in MIA PaCa2 tumor-bearing mice by treatment of albumin drug conjugates. (n=6) (C) Antitumor efficacy of albumin drug conjugates in syngeneic mT4-2D tumor-bearing mice. Treatment started after tumor volume reached ˜150 mm³. The dosing schedule was the same as the schedule in (A). MMAE, MMAE-MAL, and mouse ALDC1 were dosed at 0.5 mg/kg. Mouse ALDC1-H was dosed at 0.9 mg/kg. (n=6, data were presented as mean±SEM) All doses were shown as MMAE equivalent amount. (*p<0.05, **p<0.01, **** p<0.0001)

FIGS. 9A-9D: (A) Dose responsive curves of MMAE in MIA PaCa2 cells, PANC1 cells, and HUVEC cells. Dose responsive curves of ALDC1 and ALDC3 in (B) MIA PaCa2 cells, (C) PANC1 cells, and (D) HUVEC cells. (n=3) Data shown as mean±standard deviation. (n=3)

FIGS. 10A-10B: (A) MIA PaCa2 cells and (B) PANC1 cells were seeded in 12-well plates at 5×10⁵ cells/well and allowed to attach overnight. Cells were first starved in serum free medium for 2 hours followed by treatment with macropinocytosis inhibitor (EIPA: 5-(N-Ethyl-N-isopropyl)amiloride) for 30 min. The cells were incubated in serum free medium containing 20 μM fluorescein-albumin conjugate (ALDC1) for another 2 hours. Then cells were detached and analyzed by flow cytometer (Accuri). EIPA treatment at 50 μM and 75 μM could significantly inhibit the uptake of ALDC1 in both cell lines, indicating the macropinocytosis was involved in the endocytosis process. (*p<0.05, **p<0.01)

FIG. 11: Standard curves for LC-MS quantification of MMAE.

FIG. 12: IVIS imaging of tumor-bearing mice after administration of a single dose of Cy7-rHSA (Cy7: rHSA=1:1) and Cy7 (3)-rHSA (Cy7: rHSA=3:1) at 1 mg/kg (Cy7 equivalent amount) through tail vein injection. (Left: back view; right: ventral view).

FIGS. 13A-13G: Body weight change of mice when dosed with highest tolerated dose. PBS, MMAE, ALDC1 and ALDC3 were dosed at Day 0, 4, 8, and 12. Body weights of mice during and after treatments were monitored. 100 μL of PBS was dosed intravenously (n=4). MMAE was dosed at (B) 0.5 mg/kg (n=4) and (C) 0.7 mg/kg (n=5). ALDC1 was dosed at (D) 0.9 mg/kg (n=4) and (E) 1.1 mg/kg (n=3). ALDC3 was dosed at (F) 0.5 mg/kg (n=5) and (G) 0.7 mg/kg (n=5). All dosed were shown as MMAE equivalent amount. Data shown as mean±standard deviation.

FIG. 14: Plasma concentration of free MMAE after a single intravenous injection of MMAE, MMAE-MAL, ALDC1 and ALDC3 containing 0.5 mg/kg MMAE. Data shown as mean±SEM (n=3). Note that the initial time point is 10 minutes (0.167 hours) and not 0 minutes. Y-axis is in linear scale. Pre-mixing can protect the and delay premature cleavage of drug from the albumin carrier

FIGS. 15A-15E: Body weight change of mice when dosed with 0.9 mg/kg MMAE equivalent dose. PBS (A), MMAE-MAL (C), ALDC1 (D), and ALDC3 (E) were dosed at Day 0, 4, 8, and 12. Body weights of mice during and after treatments were monitored. (n=5). Data shown as mean±standard deviation. Mice in MMAE group (B) were dosed with 0.9 mg/kg MMAE at Day 0 and 4. On Day 6, mice in MMAE group were euthanized due to significant body weight loss (>20%). Mice in MMAE-MAL group were euthanized on Day 12 due to significant body weight loss. Other groups were euthanized on Day 13.

FIGS. 16A-16B: Body weight change of (A) MIA PaCa2 tumor-bearing mice and (B) syngeneic mT4-2D tumor-bearing mice during dosing. Dosing groups and schedules were same as described in FIG. 8. Data shown as mean±standard deviation.

FIG. 17: Regression of pancreatic tumors in immunocompetent mice with pre-mixed drug-albumin conjugate. Pre-mixing low dose and high-dose show tumor regression over prodrug that binds albumin in blood. Pre-mixing is better than having prodrug bind to circulating albumin in blood. Data shown as mean±SEM.

FIG. 18: Pre-mixed drug-albumin conjugate beats out Gemcitabine (Gem) and Abraxane +Gem combination (i.e., first-line treatment for patients with advanced pancreatic cancer). ALDC1 achieves significant delay in tumor growth compared to Abraxane+gemcitabine. Interestingly no difference between ALDC at high dose and combination of ALDC at high dose and gemcitabine. Pre-mixing drug to albumin allows for higher dosing, which is more effective. Data shown as mean±SEM. * p<0.1, ** p<0.01, **** p<0.0001; two-way ANOVA followed by Tukey's multiple comparisons.

FIG. 19: Improved selectivity of ALDC1 for tumors compared to Abraxane (nab-paclitaxel). *calculated from Li et al., 2014. International Journal of Pharmaceutics 468.1-2 (2014): 15-25. ALDC1 released more active drug in tumor tissue relative to healthy tissue (e.g., lung, liver, and spleen). The ratio of delivery of active drug in tumor to tissue is much higher than the amount of paclitaxel released from nab-paclitaxel (Abraxane) into subcutaneous pancreatic xenograft tumor tissues.

### DESCRIPTION OF ILLUSTRATIVE EMBODIMENTS

To harness the intrinsic transport properties of albumin yet improve the therapeutic index of current in situ albumin-binding prodrugs, the present studies concerned albumin-drug conjugates with a controlled loading that achieved better antitumor efficacy. Model drug monomethyl auristatin E (MMAE) was conjugated ex vivo to Cys34 of albumin via a cathepsin B-sensitive dipeptide linker to ensure that all drug would be bound specifically to albumin. The resulting albumin-drug conjugate with a drug to albumin ratio (DAR) of 1 (ALDC1) retained the native secondary structure of albumin compared to conjugate with a higher DAR of 3 (ALDC3). ALDC1 exhibited improved drug release and cytotoxicity compared to ALDC3 in vitro. Slower plasma clearance and increased drug exposure over time of ALDC1 were observed compared to ALDC3 and MMAE prodrug. In single dose studies with MIA PaCa2 xenografts, cohorts treated with ALDC1 had the highest amount of MMAE drug in tumor tissues compared to other treatment arms. After multiple dosing, ALDC1 significantly delayed the tumor growth compared to control treatment arms MMAE, MMAE-linker conjugate and ALDC3. When dosed with the maximum tolerated dose of ALDC1, there was complete eradication of 83.33% of the tumors in the treatment group. Ex vivo conjugated ALDC1 also significantly inhibited tumor growth in an immunocompetent syngeneic mouse model that recapitulates the phenotype and clinical features of human pancreatic cancers. Thus, site-specific loading of drug to albumin at 1:1 ratio allowed the conjugate to maintain the native structure of albumin and its intrinsic properties. By conjugating the drug to albumin prior to administration minimized premature cleavage and instability of the drug in plasma and enabled higher drug accumulation in tumors compared to in situ albumin-binding prodrugs. This strategy to control drug loading ex vivo ensures complete drug binding to the albumin carrier and achieves excellent antitumor efficacy, and it has the potential to greatly improve the outcomes of anticancer therapy.

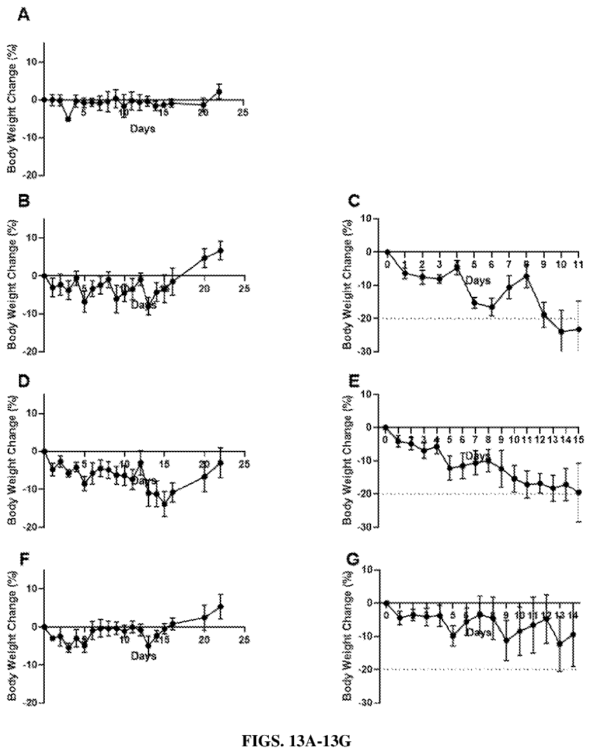

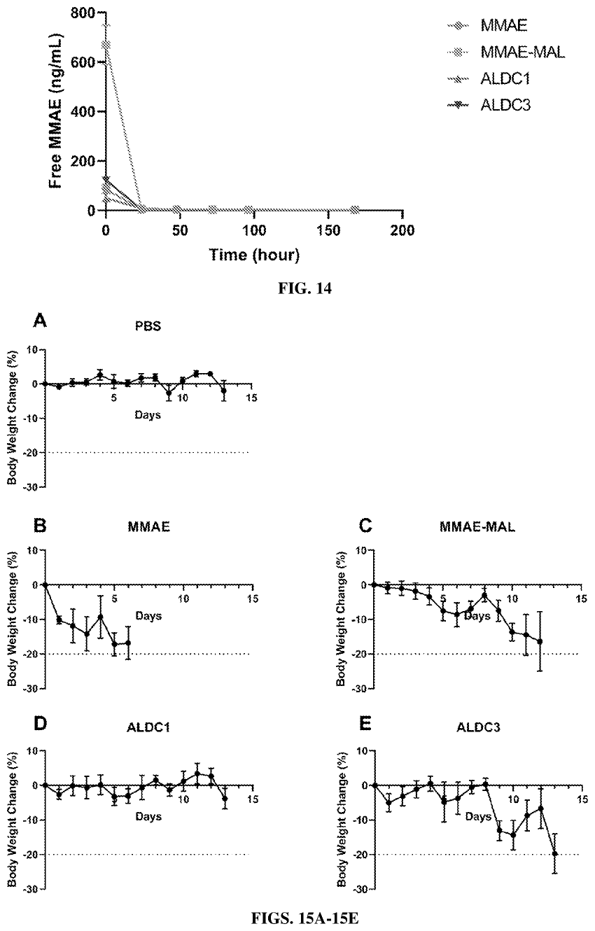

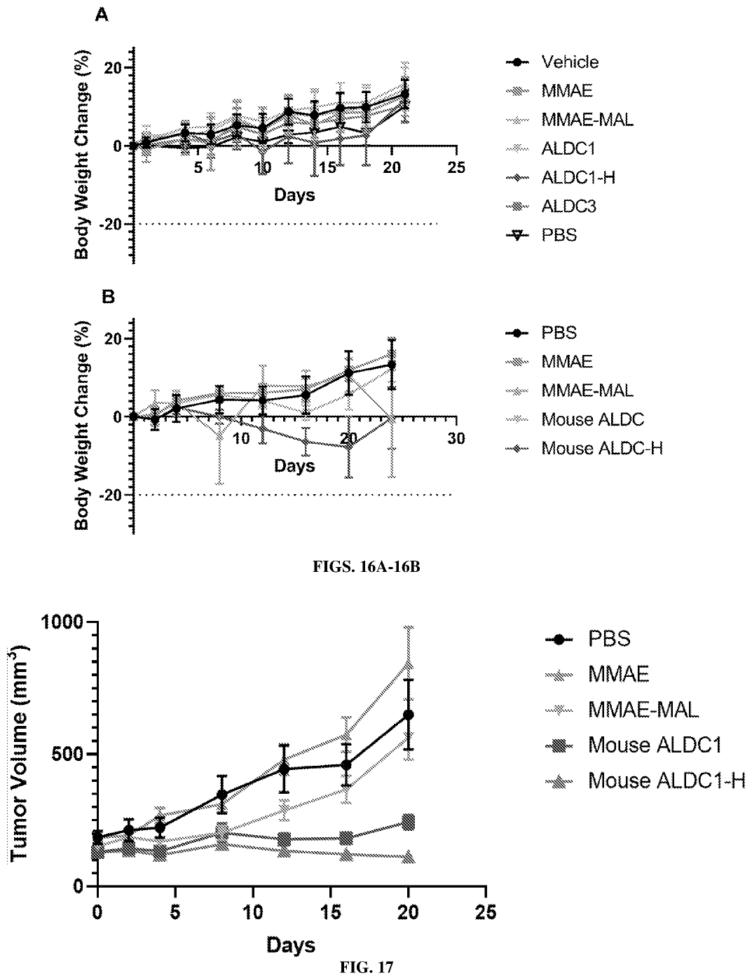

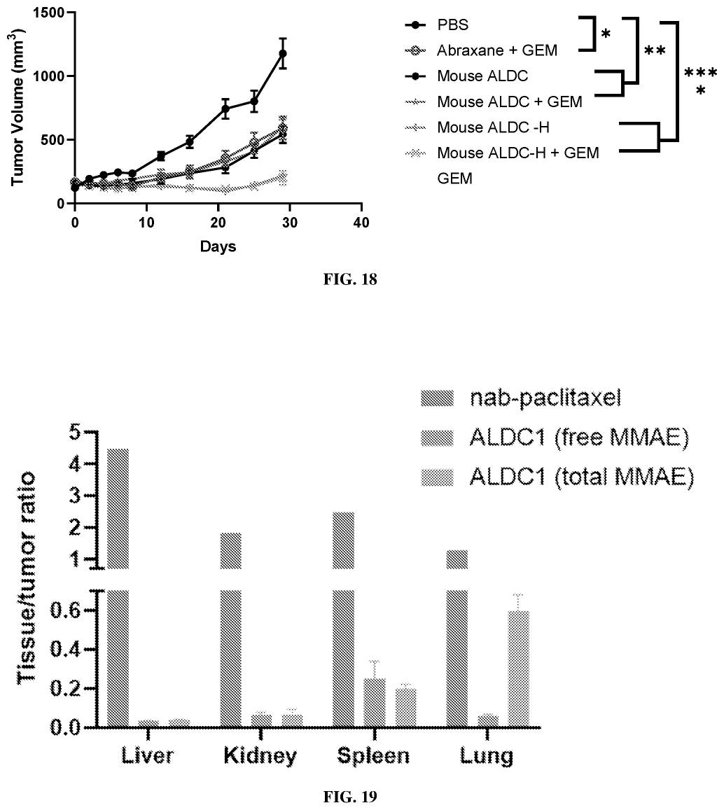

Specifically, the present in vivo studies showed that the ex vivo conjugation of the drug to the albumin surprisingly resulted in increased efficacy. FIG. 13 shows that the MMAE-MAL prodrug that binds to circulating endogenous albumin has a lower maximum tolerated dose (shown as percent weight loss from original weight) than ALDC1. Thus, a higher concentration dose of ALDC1 (and thus more MMAE-MAL bound ex vivo to the albumin) can be administered than MMAE-MAL that binds to endogenous albumin. This is important for dosing and minimizes premature cleavage of prodrug to the active drug. FIG. 14 shows that the prodrug MMAE-MAL that binds to circulating endogenous albumin is cleaved prematurely to active MMAE, whereas the ALDC1 that involves ex vivo loading of the MMAE-MAL prodrug to albumin at 1:1 stoichiometric ratio ‘retains’ the prodrug better and does not prematurely release MMAE. In addition, the loading of the linker-prodrug with albumin ex vivo allowed for the prodrug to be less susceptible to premature cleavage in the active form, compared to the prodrug that is delivered in vivo and binds to the circulating endogenous albumin. This affected the pharmacokinetics (Table 1), the drug stability in blood (FIG. 14), and indirectly the mouse weight loss with prodrug compared to ALDC1 when administered at equivalent drug dose (FIG. 15C vs. 15D) and the differences in efficacy (FIG. 17).

Accordingly, in certain embodiments, the present disclosure provides methods for the production of albumin drug conjugates by pre-mixing ex vivo. Pre-mixing can allow for higher dosage to reach the tumor site and better antitumor efficacy as compared to having a prodrug bind to circulating endogenous albumin in vivo. The drug may be conjugated to to Cys34 of albumin by a cleavable linker, such as a cathepsin B sensitive valine-citrulline dipeptide linker. The albumin and drug may be conjugated at a 1:1 ratio. The drug may be an anti-cancer agent, such as a chemotherapeutic agent. Further provided herein are methods for the treatment of cancer by administering an effective amount of the albumin drug conjugate provided herein.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

The term “substantially free of” is used to 98% of the listed components and less than 2% of the components to which composition or particle is substantially free of.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

“Treatment” and “treating” refer to administration or application of a therapeutic agent to a subject or performance of a procedure or modality on a subject for the purpose of obtaining a therapeutic benefit of a disease or health-related condition.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The terms “contacted” and “exposed,” when applied to a cell, are used herein to describe the process by which a construct and a therapeutic agent are delivered to a cell or are placed in direct juxtaposition with the target cell.

The term “drug” refers to a therapeutic agent that can be conjugated to a linker that covalently binds to a free thiol of albumin. For example, the drug may be a chemotherapeutic agent that is conjugated to a cleavable linker.

### II. METHODS OF TREATMENT

Further provided herein are methods for treating or delaying progression of cancer in an individual comprising administering an effective amount of an albumin drug conjugate, such as an albumin chemotherapeutic conjugate. The drug may be any therapeutic or diagnostic agent.

A “therapeutic agent” as used herein refers to any agent that can be administered to a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

A “diagnostic agent” as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and/or hairy cell leukemia.

In some embodiments, the subject is a mammal, e g, a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

Therapeutically effective amounts of the compound can be administered by a number of routes, including parenteral administration, for example, intravenous, intraperitoneal, intramuscular, intrasternal, or intraarticular injection, or infusion. The therapeutically effective amount of the compound is that amount that achieves a desired effect in a subject being treated. For instance, this can be the amount of the compound necessary to inhibit advancement, or to cause regression of viral disease, or which is capable of relieving symptoms caused by viral disease.

The compound can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of the compound will be dependent on the subject being treated, the severity and type of the affliction, and the manner of administration. The exact amount of the compound is readily determined by one of skill in the art based on the age, sex, and physiological condition of the subject. Effective doses can be extrapolated from dose-response curves derived from in vitro or animal model test systems.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain embodiments, the compositions and methods of the present embodiments involve an albumin drug in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The albumin drug conjugate may be administered before, during, after, or in various combinations relative to an additional cancer therapy, such as immune checkpoint therapy. The administrations may be in intervals ranging from concurrently to minutes to days to weeks. In embodiments where the albumin drug conjugate is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below albumin drug conjugate is “A” and an additional anti-cancer therapy is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Immunotherapy

Various immunotherapies are known that may be used, including, e.g., anti-PD1 antibodies or compounds, anti-PD-L1 antibodies or compounds, anti-CTLA-4 antibodies or compounds, OX40 agonists, IDO inhibitors, anti-GITR antibodies or compounds, anti-LAGS antibodies or compounds, anti-TIM3 antibodies or compounds, anti-TIGIT antibodies or compounds, and anti-MERTK antibodies or compounds, an oncolytic virus immunotherapy, intratumoral injections; immunotherapies targeting STING, NLRP3, TLR9, CPG, TLR4, LTR7/8, OX40, or MER-tk; an anti-CTLA-4, anti-PD1, anti-PDL1, or anti-CD40 immunotherapy; FLT-3-ligand immunotherapies, and/or IL-2 cytokine immunotherapies. Additionally, the albumin drug conjugate could be combined with cell therapies, such as T cells, NK cells, or dendritic cells that may be engineered to express a CAR or TCR.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144 (Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311).

In some embodiments, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy. In one aspect, the adoptive T cell therapy comprises autologous and/or allogenic T cells. In another aspect, the autologous and/or allogenic T cells are targeted against tumor antigens.

2. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

3. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Screening and Characterization of Albumin-Drug Conjugates

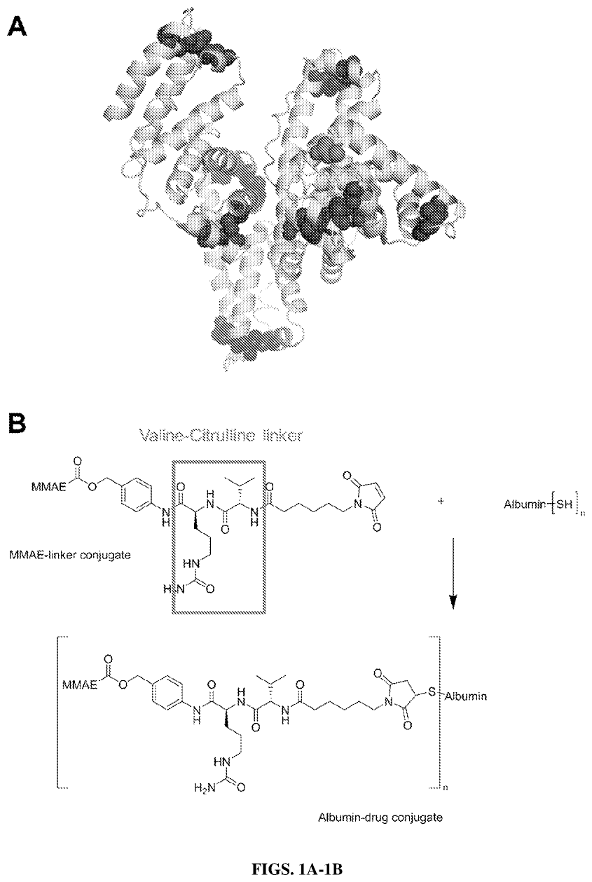

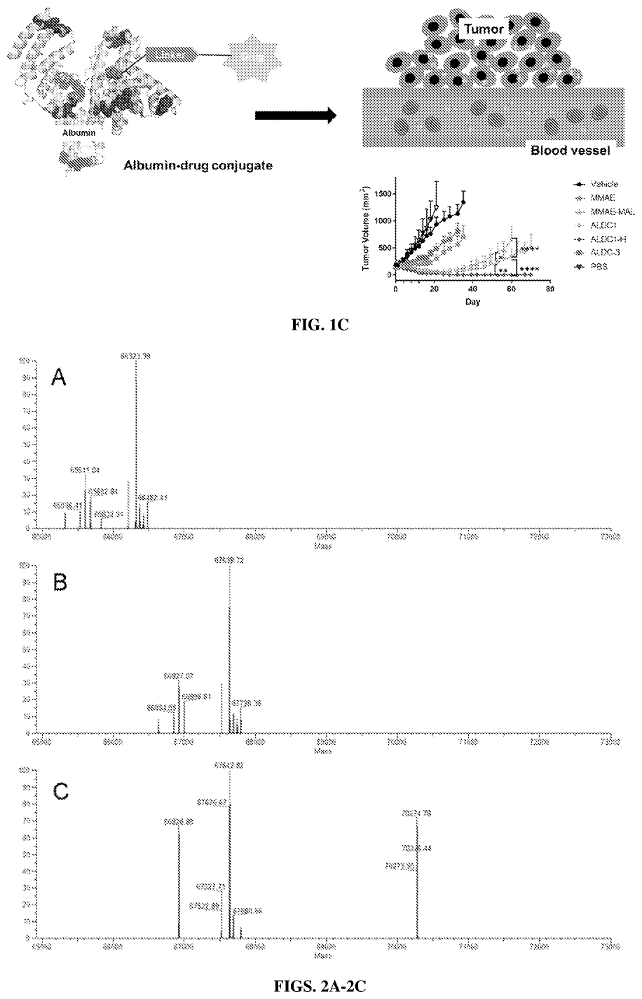

In this study, a drug-linker conjugate was used to covalently and directly react with free sulfhydryl in recombinant serum albumin. The MMAE-linker prodrug conjugate (MMAE-MAL) contains a thiol-reactive maleimide group, a protease sensitive valine-citrulline dipeptide linker and a p-aminobenzyl carbamate (PABC) spacer (FIG. 1B). For albumin-drug conjugate containing one drug per albumin molecule (ALDC1), the maleimide group of the drug-linker conjugate reacts with the single, free thiol of cysteine 34 of albumin (FIG. 1A). The conjugation of drug to albumin was monitored by the measuring the decreased amount of available free thiol in albumin. As shown in Table 1, the average number of free thiols per albumin in ALDC1 was about 1 before conjugation and close to 0 afterwards. The process was also validated by the increased molecular weight of albumin From the mass spectra, there was a shift in the molecular weight of ALDC1 from albumin alone, with an increase in the amount of the molecular weight of a single drug-linker conjugate (FIGS. 2A and 2B). The conjugation was site-specific and homogeneous. For the synthesis of albumin-drug conjugate with multiple payloads (ALDC3), a controlled reduction by TCEP was first used to expose more reactive thiols from disulfide bonds on albumin (FIG. 1A) for conjugation. The average number of free thiols per albumin after reduction was about 3 (Table 1). After conjugation, the shift in the molecular weight was equal to one or multiple amounts of the drug-linker conjugates (FIG. 2C). These results collectively indicate successful conjugation through the reaction of maleimide and thiol groups.

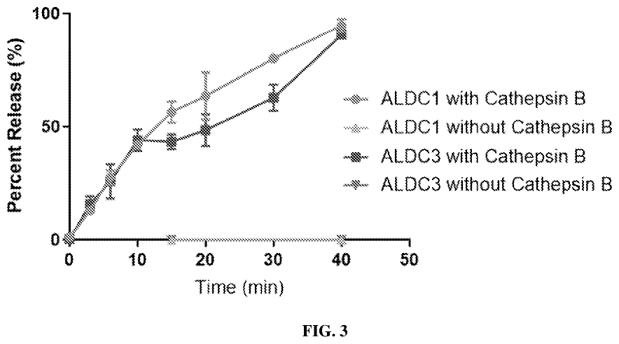

The valine-citrulline dipeptide linker (FIG. 1) was designed to be cleaved by cathepsin B enzyme after uptake by targeted cells, while maintaining the stability of the albumin-drug conjugate in blood circulation. To evaluate the release profile of albumin-drug conjugates, ALDC1 and ALDC3 were incubated in an acidic buffer with or without Cathepsin B, both containing equal amount of MMAE as shown in FIG. 3. When incubated without Cathepsin B, the released MMAE from ALDC1 and ALDC3 was not detectable during the investigated period, indicating that the albumin-drug conjugates were stable without Cathepsin B. Upon incubation with Cathepsin B, ALDC1 and ALDC3 released 94.73% and 90.62% of the conjugated MMAE within 40 min, respectively. Initially, the cleavage rate of the linker by Cathepsin B was similar, as shown in FIG. 3. However, the cleavage rate of ALDC3 slowed down after 10 min and then gradually increased. This may be due to the heterogenous conjugation of ALDC3. With ALDC3, there are on average 2 more free thiols where the location cannot be controlled, besides the fixed Cys34 position; as a result, there can be multiple species of albumin that have different amounts of available thiols and thus, different amounts of conjugated MMAE.

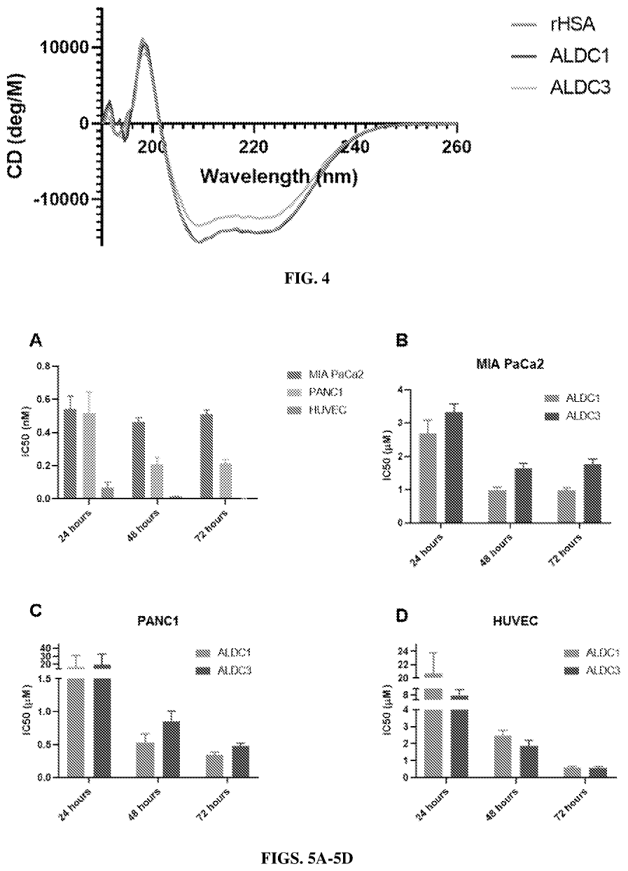

The conformational structure of albumin-drug conjugates was then characterized by circular dichroism (CD) spectroscopy. As shown in FIG. 4, minima ellipticity peaks were observed at 208 and 222 nm.[27] The spectra of ALDC1 aligns with the spectra of native albumin, indicating that the secondary structure of albumin has not been altered after conjugation of a single payload to Cys34. However, the secondary structure of ALDC3 showed a different CD spectrum in comparison to albumin and ALDC1 (FIG. 4). Controlled reduction was used to expose a limited number of thiols from the total 17 disulfide bonds present in albumin (FIG. 1A). Most of the disulfide bonds were located in the α-helix region of albumin Though the reaction was controlled, the number and location of exposed free thiols were heterogenous, as supported by the LC-MS spectra of ALDC3 (FIG. 2). Partial reduction and subsequent addition of multiple drugs to albumin may impact the structure of albumin, which has been reflected in changes to the secondary structure of ALDC3 (FIG. 4).

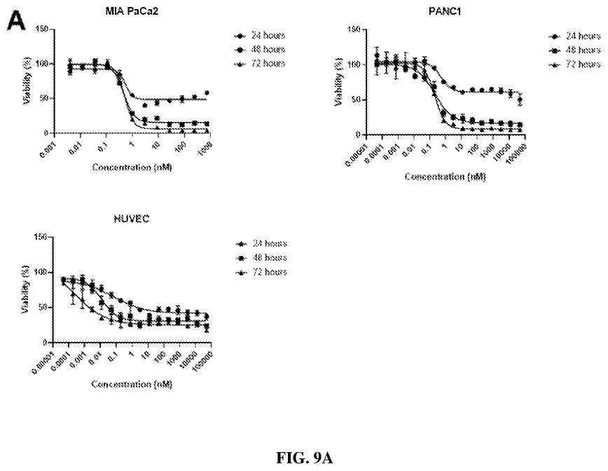

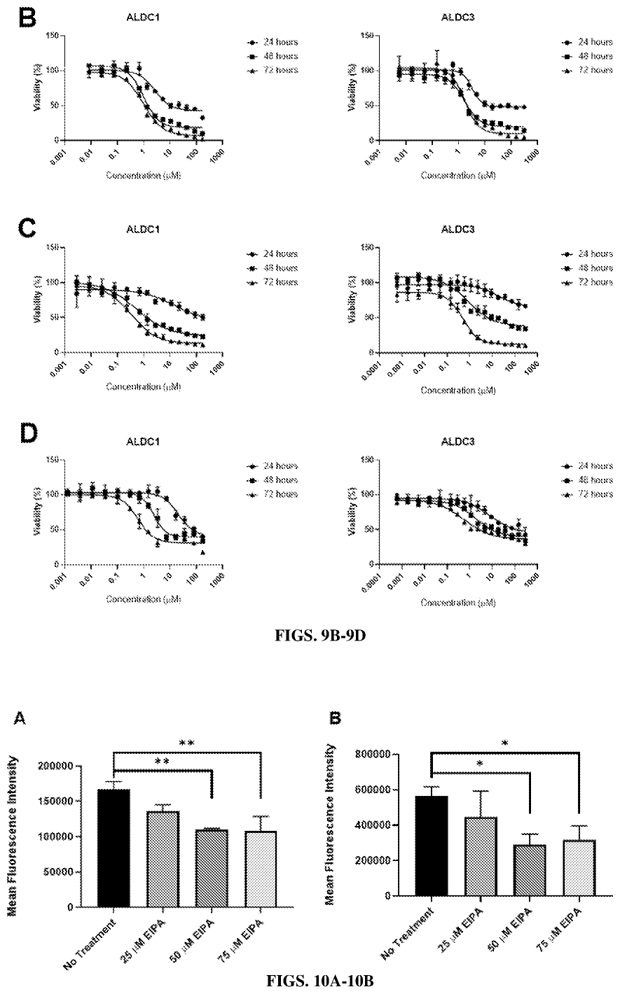

In vitro cell toxicity assessment: The toxicity of free drug and albumin-drug conjugates were evaluated in two human pancreatic cancer cell lines MIA PaCa2 and PANC1. Dose responsive curves are shown in FIG. 9. IC50 values were compared to the concentration of free MMAE. As shown in FIG. 5A, the IC50 of free MMAE in MIA PaCa2 cells was 0.54 nM, 0.46 nM, and 0.5 nM at 24, 48, and 72 hours, respectively. The IC50 of free MMAE in PANC1 cells was 0.52 nM, 0.21 nM, and 0.21 nM at 24, 48, and 72 hours, respectively. Free MMAE was more toxic to noncancerous HUVEC cells; the IC50 was 67 pM, 12 pM and 0.3 pM at 24, 48, and 72 hours, respectively. After MMAE conjugation to albumin, the IC50 of ALDC1 and ALDC3 was higher than free MMAE, exhibiting μM toxicity in MIA PaCa-2 and PANC1 cells (FIGS. 5B and 5C). The potency of the drug decreased after conjugating to albumin Interestingly, the IC50 for the free drug is better, i.e., lower, than IC50 of albumin-drug conjugate (ALDC1) in cell culture. However, in vivo the drug killing was soen to be better with ALDC1 than with the free drug. Since MMAE is a lipophilic molecule, it is able to freely diffuse and permeate across cell membranes in vitro. After MMAE conjugation to albumin, the resulting ALDC can only be internalized into cells via vesicle mediated endocytosis, such as macropinocytosis (FIG. 10). Even though MMAE can be released from ALDCs into its active form in the presence of intracellular cathepsin B, the initial uptake and release process of ALDCs was likely slower than free diffusion of MMAE, resulting in decreased potency compared to the parent drug MMAE.

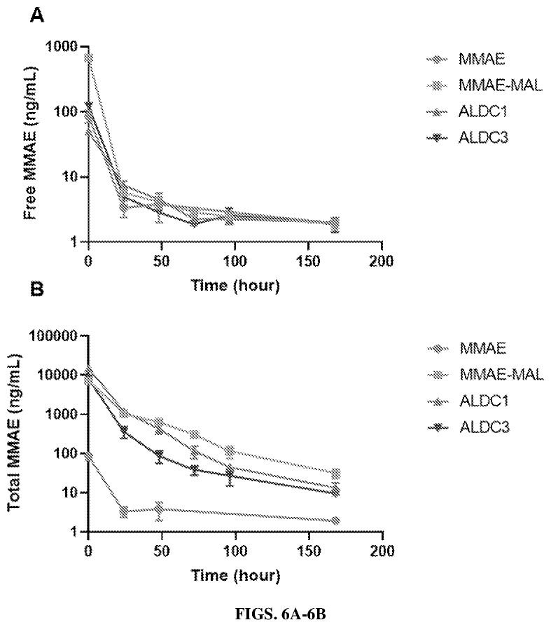

Pharmacokinetics of albumin-drug conjugates: The pharmacokinetic profiles of free MMAE and total MMAE in plasma were measured by LC-MS in MIA PaCa2 xenografts. Equivalent amounts of MMAE from various treatment arms were administered at a single dose to each group of mice (0.5 mg/kg). Total MMAE in plasma, which was from released MMAE and conjugated MMAE, was measured by LC-MS. The standard curves are shown in FIG. 11. The pharmacokinetic profiles of free and total MMAE are shown in FIGS. 6A and 6B, respectively. In FIG. 6A, the plasma concentration of free MMAE from ALDC1 group was 49.99±3.88 ng/mL 10 min after dosing, which is less than the MMAE-dosed group (86.03±17.78 ng/mL). However, the free MMAE released from MMAE-MAL prodrug and ALDC3 groups was 669.67±78.94 ng/mL and 121.74±13.74 ng/mL, respectively, which was both higher than the MMAE-dosed group. There was significant premature release of MMAE in MMAE-MAL group (13-fold) and ALDC3 group (2.4-fold) compared to the ALDC1 group, which suggests that the MMAE-MAL prodrug and ALDC3 groups are unstable in plasma.

The pharmacokinetic parameters of total MMAE (Table 1) were calculated using noncompartmental analysis (PK Solver software).[28] The AUC (area under the curve, i.e. total drug exposure in plasma over time) of total MMAE in ALDC1 group was significantly higher, which was 160% of that in MMAE-MAL group and 200% of the ALDC3 group. This finding can be due to premature cleavage and rapid clearance of MMAE in the MMAE-MAL and ALDC3 groups (shown in FIGS. 6A and 6B). Here, in both MMAE-MAL and ALDC3 arms, MMAE was more suspectable to be cleaved and prematurely released in the plasma, resulting in faster clearance than the ALDC1 group (Table 1). MMAE-MAL was designed to bind to endogenous albumin after administration. It is feasible that after entering the circulation, the exposed drug-linker conjugate was more suspectable to cleavage before it could bind to endogenous albumin. After conjugation ex vivo, the albumin-drug conjugate was more stable in plasma and only cleaved after tumor uptake. Thus, ALDC1 has better pharmacokinetics—greter area under the curve and half-life and lower clearance from tissues than MMAE-MAL (in situ binding albumin prodrug) and ALDC3 (three prodrugs per albumin molecule).

TABLE 1
Pharmacokinetic parameters of total MMAE in plasma.

| Parameters | Unit | MMAE-MAL | ALDC1 | ALDC3 |
|---|---|---|---|---|
| AUC ₀₋ₜ | ng/ml*h | 139928.1 | 224965.4 | 110343.7 |
| AUC _{0-∞} | ng/ml*h | 141193.8 | 225602.6 | 111001.4 |
| AUMC _{0-∞} | ng/ml*h\hat{ }2 | 2827552.1 | 1819599.5 | 728096.2 |
| MRT | h | 20.02 | 8.06 | 6.55 |
| T_{1/2} | h | 27.82 | 32.95 | 28.63 |
| Cl | (mg/kg)/(ng/ml)/h | 3.54E−06 | 2.2163E−06 | 4.5E−06 |

Abbreviations: AUC ₀₋ₜ: area under the zero moment curve from time 0 to 168 hours; AUC _{0-∞}: area under the zero moment curve from time 0 to infinity; AUMC 0_{0-∞}: area under the first moment curve from time 0 to infinity; MRT: mean residence time; T_{1/2}: half-life; Cl: clearance.

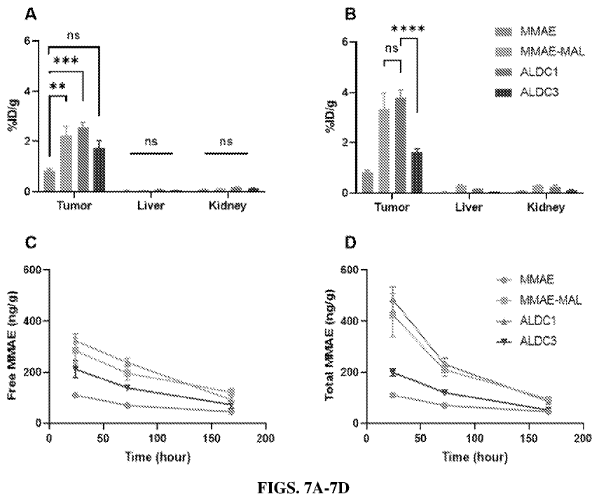

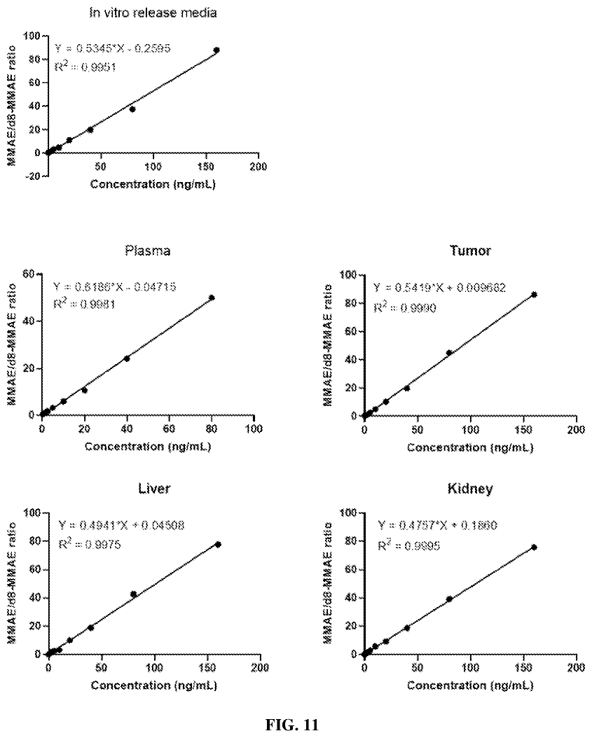

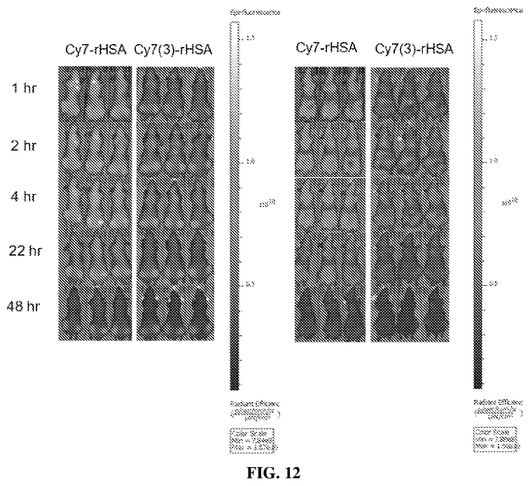

Tissue distribution: Next, the distribution of MMAE delivered in free drug form and albumin-drug conjugates were measured in select tissues (FIG. 7). In each group, mice were given a single dose of equivalent MMAE and after 24 hours dosing, released MMAE and total MMAE were both measured in homogenized tissues using a highly sensitive LC-MS method. In FIG. 7A, accumulation of free MMAE in tumor was significantly increased in ALDC1 group with a percentage injected dose per gram tissue of 2.6% ID/g. Accumulation of free MMAE in MMAE-MAL group was slightly lower (2.2% ID/g), although the difference was not statistically different (p>0.1). The amount of free MMAE in ALDC1 and MMAE-MAL groups were about 2-fold more compared to the MMAE group. ALDC3 did not improve the accumulation of free MMAE in tumor compared to MMAE group (no statistical difference). In the liver and kidney, there were low levels of free MMAE in MMAE-MAL, ALDC1 and ALDC3 groups, with no statistical difference (p>0.1) compared to MMAE group. FIG. 7B showed the tissue distribution of total MMAE. ALDC1 accumulation in tumor was the highest (3.8% ID/g) amongst all groups. For MMAE-MAL and ALDC3, the total MMAE % ID/g in tumor were lower, with 3.3% and 1.6%, respectively. Significantly more total MMAE was found in liver (0.3% ID/g) of the MMAE-MAL group than the ALDC1 group (0.2% ID/g). The differences between free MMAE and total MMAE in healthy tissues indicate that albumin-drug conjugates were still circulating in these tissues in the conjugated form. They were stable in normal tissues but can be efficiently cleaved and released MMAE at the tumor site. Thereby, significant improvement of accumulation of MMAE in tumor was observed. At 24 h after a single-dose administration, there is no significant difference in drug accumulation between MMAE-MAL prodrug and ALDC1; yet over time, there are differences in pharmacokinetics between groups. In tumor-bearing mice in different treatment arms, we measured the amount of free MMAE and total MMAE present in tumor tissue at different timepoints and calculated the amount over time. As shown in FIGS. 7C and 7D, high concentrations of free MMAE and total MMAE in tumor were observed in ALDC1 treated group. When comparing the calculated AUC_{0-168 h} values in tumor, there were ˜10% more exposure of free MMAE and ˜9.3% more exposure of total MMAE in ALDC1 treated group compared to MMAE-MAL group. And there were ˜1.5-fold higher exposure of free MMAE and ˜2-fold higher exposure of total MMAE in ALDC1 group compared to ALDC3 group. These data suggest that greater amounts of drug accumulate in tumors after administration of ALDC1 compared to the other treatment arms. To visualize the distribution of albumin-drug conjugates in tumor, maleimide activated cyanine 7 (Cy7) near-infrared fluorescent dye was conjugated to albumin as a proxy for the MMAE prodrug using the same method of ALDC1 and ALDC3 conjugation and purification. Cy7 albumin conjugates with dye to albumin ratios of 1 and 3 (Cy7(1) and Cy7(3), respectively) were injected intravenously in tumor-bearing mice for IVIS imaging. As shown in FIG. 12, strong signals were observed at tumor site, indicating the accumulation of conjugates in the tumor. The circulation signal of Cy7(3) albumin conjugate was lower than that of Cy7(1) albumin conjugate, which correlates with the tissue distribution of ALDC1 and ALDC3 albumin-drug conjugates.

TABLE 2
Determination of drug to albumin ratio (DAR) by Ellman’s
reagent (n = 3)

| | Detected thiols per albumin | | |
|---|---|---|---|
| | Before Conjugation | After conjugation | DAR |
| ALDC1 | 1.30 ± 0.06 | 0.05 ± 0.02 | 1.25 |
| ALDC3 | 3.22 ± 0.22 | 0.04 ± 0.01 | 3.18 |

In vivo antitumor efficacy: Prior to testing the efficacy of our conjugates, the maximum tolerated dose was determined for each treatment arm. To find the maximum tolerated dose, healthy mice were injected with escalating doses of either free MMAE or albumin-drug conjugates, as adapted from Hamblett et al.[14, 29] As shown in FIG. 13, the maximum doses of MMAE, ALDC1 and ALDC3 were approximately 0.5 mg/kg, 0.9 mg/kg, and 0.5 mg/kg (MMAE equivalent dose), respectively. These are the highest doses that maintained mice body weight change within 20% during the period of drug administration, using same parameters reported by Hamblett et al testing anti-CD30-vc-MMAE antibody-drug conjugate.[29] FIG. 13 shows that the MMAE-MAL prodrug that binds to circulating endogenous albumin has a lower maximum tolerated dose (shown as percent weight loss from original weight) than ALDC1. Thus, a higher concentration dose of ALDC1 (and thus more MMAE-MAL bound ex vivo to the albumin) can be administered than MMAE-MAL that binds to endogenous albumin. This is important for dosing and minimizes premature cleavage of prodrug to the active drug. FIG. 14 shows that the prodrug MMAE-MAL that binds to circulating endogenous albumin is cleaved prematurely to active MMAE, whereas the ALDC1 that involves ex vivo loading of the MMAE-MAL prodrug to albumin at at 1:1 stoichiometric ratio ‘retains’ the prodrug better and does not prematurely release MMAE.

All cohorts were further treated with the same 0.9 mg/kg MMAE equivalent dose (i.e. the maximum tolerated dose of ALDC1) and measured changes in body weight loss amongst all groups, as shown in FIG. 15. The cohort dosed with MMAE-MAL in situ albumin binding prodrug demonstrated ˜20% loss of body weight after multiple dosing and had to be sacrificed on day 12 prior to that day's dosing (FIG. 15C), whereas the ex vivo conjugated ALDC1 group demonstrated minimal weight change and could be repeatedly dosed for the whole duration of the study (FIG. 15D). The group dosed with ALDC1 exhibited signs of less toxicity than groups dosed with free MMAE, prodrug, or ALDC3. Thus, FIG. 15 shows that each treatment (except PBS) had the same amount of MMAE that was administered. The MMAE-MAL prodrug that binds to circulating endogenous albumin was more ‘toxic’ than ALDC1 (i.e, there is a greater percent weight loss from original weight) than ALDC1. Mice treated with ALDC1 had negligible changes in body weight compared to the other arms, which had increased loss in weight, especially mice treated with MMAE-MAL. This indicates more MMAE (active and prodrug) can be delivered without animal weight change by ex vivo conjugation to albumin compared to MMAE-MAL that binds to albumin in situ.

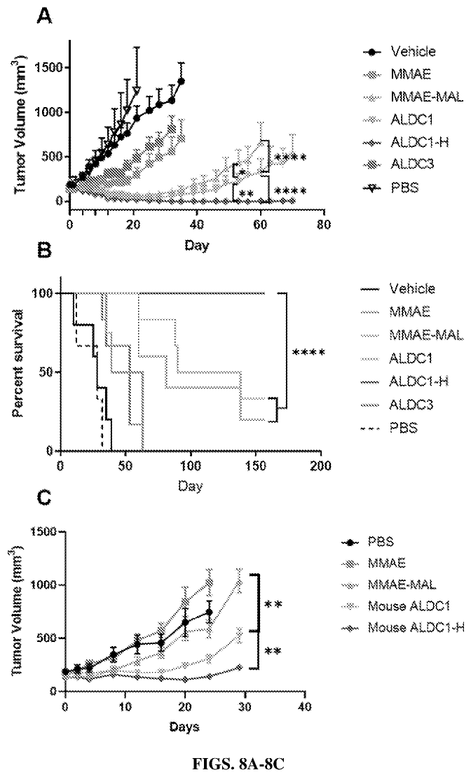

The antitumor efficacy of albumin-drug conjugates was first evaluated in MIA PaCa2 xenografts. MIA PaCa2 cells are human pancreatic cancer cells with typical genetic alterations that are common in pancreatic ductal adenocarcinoma (i.e. possess KRAS mutation, have p53 mutation, and contain a CDKN2A homozygous deletion).[30] Recent work also suggests that this cancer cell line actively scavenges albumin, which can be leveraged for delivery.[31-33] Here, tumor-bearing mice were dosed with either MMAE, MMAE-MAL, ALDC1 or ALDC3 every 4 days for a total of 4 doses. Each treatment arm had an equivalent amount of MMAE (0.5 mg/kg). Since ALDC1 exhibited a higher maximum tolerated dose than the other groups, we an additional group was included that was dosed with ALDC1 at a higher concentration of 0.9 mg/kg MMAE, (denoted as ALDC1-H group). No significant body weight loss was observed during the efficacy study when dosing at select tolerated doses (FIG. 17). As shown in FIG. 8A, free MMAE slowed down tumor growth compared to vehicle and PBS control groups. When delivering same dose of MMAE using MMAE-MAL and ALDC1, the tumor growth was significantly delayed compared to free MMAE and controls. At 60 days, ALDC1 significantly retarded tumor growth compared to the MMAE-MAL treatment arm. However, at higher DAR of 3, ALDC3 only had a similar effect as free MMAE to slow tumor growth. The median survival of tumor-bearing mice dosed with MMAE-MAL, ALDC1 and ALDC3 (0.5 mg/kg equivalent to MMAE) was 81, 114 and 53 days, respectively (FIG. 8B). Interestingly, when dosed with 0.9 mg/kg ALDC1 (ALDC1-H), tumors shrink to unmeasurable sizes after 4 doses and are eradicated completely in 5 of the 6 mice; this tolerated dose dramatically improved the survival ratio of mice. Mice given ALDC1 at 0.9 mg/kg had 100% survival even at 157 days, which is 145 days after last treatment.

Next, the efficacy of the ex vivo pre-conjugation strategy was further validated in a syngeneic mouse model of pancreatic cancer Immunocompetent mice harboring mT4-2D pancreatic tumors were dosed with the conjugates and compared with free drug and the MMAE-MAL prodrug arms. Tumor growth was significantly delayed in mouse ALDC1 group and mouse ALDC1-H group compared to the MMAE-MAL group (FIG. 8C). These results strongly support the hypothesis that ex vivo conjugation at controlled drug to albumin ratios improves antitumor efficacy in vivo, even compared to conjugates at higher drug to albumin ratios and in situ albumin binding MMAE prodrug.

Further studies were performed on C57BL/6 mice bearing subcutaneous KPC tumors (i.e. mice with intact immune system having pancreatic tumors harboring main mutations: mutant KRAS and loss of p53 tumor suppressor). Pancreatic tumors in immune-competent mice are immune suppressive and thus a more realistic model of disease. After tumor formation, mice were injected at day 0, 4, 8, and 12 with treatment arms with equivalent amount of drug (except for PBS/saline and mouse ALDC1-H, which had about 2× amount of drug). FIG. 1 shows that pre-mixed drug conjugate at high dose and in combination with Gem regress tumors better than Gem and standard Abraxane+Gem. (Gem, 100 mg/kg, (intraperitoneally) IP, Day 0, 4, 8, 12, and 16). Thus, pre-mixing was shown to have higher efficacy than having the prodrug bind to circulating albumin the blood.

A highly potent drug MMAE was pre-conjugated to albumin through a protease-sensitive dipeptide linker for antitumor drug delivery. Controlled, site-specific loading of drug to albumin at a 1:1 molar ratio significantly improved efficacy, whereas there was no therapeutic benefit by increasing the drug to albumin ratio. By maintaining a DAR of 1 through site-specific conjugation at Cys34 (ALDC1), there was negligible effect on the native structure of albumin, and an improvement of the drug half-life and antitumor efficacy was achieved. In addition, the therapeutic window was increased by ex vivo conjugation of MMAE to albumin prior to administration to minimize premature drug release of that is experienced with the drug-linker conjugate (MAME-MAL) designed to bind to endogenous albumin. The delivery of intact albumin-drug conjugates showed excellent antitumor efficacy in tumor-bearing mice, with noticeable long-lasting tumor regression and improved overall survival. Since albumin is able to transcytose across the vascular endothelium, the carrier may not necessarily be dependent on the heterogeneous enhanced permeation and retention effect in tumors to achieve drug accumulation in tumors.

### Example 2—Methods and Materials

Synthesis of albumin-drug conjugates at different ratios: To synthesize albumin-drug conjugate with a drug to albumin ratio (DAR) of 1 (denoted as ALDC1), recombinant human serum albumin (rHSA, Albumin Biosciences) or recombinant mouse serum albumin (rMSA, Albumin Biosciences) was initially dissolved in phosphate buffered saline (PBS, pH 7.4) to make a 10 mg/mL solution. Drug-linker conjugate (mc-vc-pab-MMAE, AstaTech) was dissolved in acetonitrile. Then, 4 volumes of either recombinant serum albumin (rSA) solution was mixed with 1 volume of drug-linker conjugate solution. The molar ratio of rSA to drug-linker conjugate was 1:3 in the final mixture. The mixture was gently mixed and incubate on ice for 1 hour. The unreacted drug-linker conjugate was removed by PD-10 column (GE Healthcare) according to the manufacturer's protocol, followed by buffer exchange with PBS using Amicon ultra centrifugal units (molecular weight cut off 30,000 Da, Millipore).

To synthesize albumin-drug conjugate with a higher DAR of 3 (denoted as ALDC3), reduced rSA was prepared to provide more accessible sites for drug conjugation. Briefly, rSA was dissolved in reducing buffer (50 mM sodium borate and 50 mM sodium chloride in water, pH 8.0) to make a 10 mg/mL solution. 2.5 mole equivalent tris(2-carboxyethyl) phosphine hydrochloride (TCEP, Millipore Sigma) was added to the solution and incubated at 37° C. for 15 min. Drug-linker conjugate was subsequently added in the solution as described above to synthesize and purify albumin-drug conjugate with a higher drug to albumin ratio.

Characterization of albumin-drug conjugates: DAR was determined using Ellman's reagent (ThermoFisher) with a molar absorptivity method according to manufacturer's protocol. rSA, reduced rSA before conjugation, and albumin-drug conjugates after conjugation were all sampled. The concentration of rSA was determined by Pierce™ BCA protein assay kit (Thermo Scientific). DAR was calculated as the following: DAR=available free thiol per albumin before conjugation—available free thiol per albumin after conjugation.

The molecular weight of synthesized ALDCs were analyzed by the University of Texas at Austin Center for Biomedical Research Support Proteomics Facility using liquid chromatography-mass spectrometry (LC-MS) on a Thermo Orbitrap Fusion Tribrid mass spectrometer with the ion trap or FT detector. A fast gradient of 0.1% formic acid/water and 0.1% formic acid/acetonitrile over 10 minutes was used to elute the intact proteins from an OPTI-TRAP™ protein microtrap (Optimize Technologies). The Orbitrap Fusion was operated in Intact Protein Mode either with the ion trap detector or the FT detector set at 15,000 resolution from 400-2000 m/z. The data was deconvoluted using Thermo Protein Deconvolution software.

Circular dichroism (CD) spectra were recorded by JASCO J-815 CD Spectrometer. Samples were diluted to 200 μg/mL in PBS at pH 7.4 and measured in a rectangular quartz cell (pathlength 1 mm, JASCO) sealed with a Teflon stopper. The CD spectra was recorded in the range from 260 nm to 190 nm with 1-nm step and 1 second sampling time.

Quantification of MMAE by LC-MS: An Agilent 1260 Infinity liquid chromatography system (G1312B) with an Agilent 6530 Q-TOF mass spectrometer was used to detect the drug MMAE. 5 μL of sample was injected into an Eclipse Plus C18 column (50×2.1 mm, 5 μm) followed by a gradient elution at 0.7 mL/min. The gradient started with 95% mobile phase A (water containing 0.1% formic acid) and 5% mobile phase B (methanol containing 0.1% formic acid), then the mobile phase A was linearly decreased to 80% in 5 min and further linearly decreased to 5% in 12 min. Electrospray ionization source was used in positive mode. To derive standard curves for MMAE, drug standards were spiked into blank matrix along with internal standard. Samples were then prepared for LC-MS analysis. MMAE was monitored at m/z 740 [M+Na]+, 719 [M+H]+, 371 [M+Na+1-1]²⁺ and 360 [M+2H]²±. Deuterated (D8) MMAE as internal standard was monitored at m/z 748 [M+Na]+, 727 [M+H]+, 375 [M+Na+1-1]²⁺ and 364 [M+2H]²±. The peak area of each drug standard was derived by the peak area of internal standard. The peak area ratios were then plotted as a function of standard concentrations and data points were fitted using linear regression (GraphPad). MMAE concentrations in test samples were quantified using the peak area ratio between MMAE and D8-MMAE and calculated using the standard curves.

Drug release of albumin-drug conjugates: Drug release of MMAE from its albumin-drug conjugate was performed using cathepsin B enzyme to cleave the self-immolative linker between the MMAE drug and albumin.[24] Cathepsin B extracted from human liver (Sigma-Aldrich) was activated in a buffer containing 30 mM DTT, 15 mM EDTA at pH 5.5 for 15 min at room temperature. The concentration of Cathepsin B in activation buffer is 0.125 μM. Albumin-drug conjugates in 25 mM sodium acetate buffer (pH 5.5) was mixed with Cathepsin B at the volume ratio of 14:1 (v/v). The target molar ratio of Cathepsin B to linker in albumin-drug conjugates was 1:1000 (mol/mol). The reaction mixture was incubated at 37° C. water bath. 10 μL sample aliquots were taken at predetermined time points and immediately quenched by adding E-64 (trans-Epoxysuccinyl-L-leucylamido(4-guanidino)butane protease inhibitor, Sigma-Aldrich) to a final concentration of 0.25 μM. Acetonitrile was added to a final concentration of 95% (v/v). The supernatant was subsequently analyzed by LC-MS to quantify released free MMAE.

Cell lines and culture: Human pancreatic cancer cell line MIA PaCa2 cells and primary umbilical vein endothelial cells (HUVEC) were purchased from American Type Culture Collection (ATCC). Human pancreatic cancer cell line PANC1 cells were kindly provided by Dr. Zhengrong Cui (College of Pharmacy, The University of Texas at Austin). Mouse mT4-2D pancreatic cancer cell line, which is derived from a Kras^{+/LSL-G12D}Tp53^{+/LSL-R172H}Pdx1-Cre transgenic model of pancreatic cancer, was kindly provided by Dr. Kyaw Aung (Livestrong Cancer Institutes, Dell Medical School, The University of Texas at Austin). MIA PaCa2 cells, PANC1, and mT4-2D cells were maintained in Dulbecco's Minimum Essential Medium with high glucose (Corning). Cell culture medium was supplemented with 10% fetal bovine serum (Gibco) and 100 U/mL penicillin-streptomycin (Gibco). HUVEC cells were maintained according to protocol provided by ATCC. All cells were maintained at 37° C. in a humidified atmosphere with 5% carbon dioxide.

MTT Assay: Cells were seeded in 96 well plates at a density of 5,000 cells/well. Cells were incubated overnight to allow attachment to the bottom of the plates. Cells were treated with MMAE and albumin-drug conjugates at various concentrations in 100 μL medium for 24 hours, 48 hours and 72 hours, respectively. Untreated cells were used as control. After the treatments, 10 μL of MTT (3-(4,5-Dimethylthiazol-2-yl)-2,5-Diphenyltetrazolium Bromide, 5 mg/mL) was added to each well and incubated for an additional 4 hours. Then, the medium was replaced with 150 μL dimethyl sulfoxide (DMSO). After the formazan crystals were completely dissolved, the absorbance at 570 nm was read by a plate reader (Infinite®200 Pro, Tecan). Cell survival rate was calculated as follows:

$$Survial rate ( \% ) = ( \frac{{Abs}_{Treatment} - {Abs}_{Blank}}{{Abs}_{Cotrol} - {Abs}_{Blank}} ) \times 100 \% .$$

The IC50 (half maximal inhibitory concentration) was calculated using GraphPad Prism 8.

Maximum tolerated dose: All animal experiments were approved and performed under the guidelines by the Institutional Animal Care and Use Committee at. The University of Texas at Austin. To determine the maximum tolerated dose of free MMAE drug and albumin-drug conjugates (ALDC1 and ALDC3), they were injected into healthy athymic female NCr nude mice (Taconic) via tail vein at MMAE equivalent doses ranging from 0.5 to 1.4 mg/kg. Mice were injected every 4 days for a total of 4 repeated doses. Body weight and general appearance of each mice were monitored for 10 days after the last dose of injection.

Tumor-bearing mouse models: Eight to ten week old athymic female NCr nude mice and C57BL/6 mice (Taconic) were used to establish xenograft mouse model and syngeneic mouse model, respectively. MIA PaCa2 and mT4-2D cells were harvested from culture and resuspended in serum free cell culture media at 4×10⁷ cells/mL and 1×10⁷ cells/mL, respectively. The cell suspension was then gently mixed with equal volume of Matrigel® (Corning). Subsequently, 100 μL of each mixed cell suspension (MIA PaCa2 and mT4-2D) was subcutaneously inoculated into both flanks of NCr nude mice and C57BL/6 mice, respectively. After the tumors were palpable, tumor volumes were measured two times a week. The tumor volume was calculated as (1/2×length×width).

Pharmacokinetics and tissue distribution: Thirty-six mice bearing MIA PaCa2 tumors were equally divided into 4 groups. A single dose treatment of either MMAE, MMAE-MAL, ALDC1 or ALDC3 was administered through tail vein injection when the tumors reached ˜500 mm³ in volume. All treatment arms were dosed with an equivalent amount of MMAE at 0.5 mg/kg. At 10 min, 24 h, 48 h, 72 h, 96 h and 168 h post-administration, blood samples were collected from 3 mice in each group through tail vein. Plasma was separated immediately using BD microtainer. At 24 h, 72 h and 168 h after administration, 3 mice from each group were euthanized to collect liver, kidney, and tumor tissue samples. Plasma and tissue samples were stored at −80° C. until further analysis.

To determine the amount of free MMAE in plasma samples, 1 μL D8-MMAE (250 ng/mL) internal control was added into 10 μL plasma samples and then mixed with 89 μL acetonitrile. The mixture was thoroughly mixed and centrifuged (12,000 rpm, 20 min, 4° C.). The supernatant was collected for LC-MS quantification. To determine free MMAE in mouse tissues, weighed tissues were homogenized with ice-cold PBS containing protease inhibitors (cOmplete™ ULTRA Tablets, Roche) using a tissue homogenizer (Fisher Scientific) to a final concentration of 600 mg/mL. The homogenized suspension was centrifuged at 12,000 rpm for 20 min at 4° C. As an internal control, 1 μL D8-MMAE (250 ng/mL) was added into 20 μL of the supernatant and then mixed with 79 μL acetonitrile. After the mixture was mixed and centrifuged, the supernatant was collected for LC-MS for quantification.

To determine total MMAE (i.e. cleaved MMAE and albumin-conjugated MMAE) in plasma and tissues, a forced degradation was done to completely release conjugated MMAE present in plasma and tissue samples.[25] Briefly, freshly prepared papain (Sigma-Aldrich) was added to plasma and tissue samples at a final concentration of 2 mg/mL, and the mixtures were incubated at 40° C. for 16 hours. The resulting samples were treated as method described above for LC-MS quantification.

In vivo antitumor efficacy: Mice bearing MIA PaCa2 xenografts and syngeneic mT4-2D C57BL/6 mice were randomized when the tumor sizes were ˜150 mm³, respectively. MMAE, MMAE-MAL, ALDC1 and ALDC3 were administered through tail vein every 4 days for a total of 4 doses in MIA PaCa2 xenografts. PBS and albumin vehicle controls were also administered. PBS control, MMAE, MMAE-MAL, mouse ALDC1 were injected intravenously every 4 days for a total of 4 doses in syngeneic mT4-2D C57BL/6 mice. Tumor sizes were measured by a digital caliper twice a week starting from Day 0. Mice were euthanized when either tumor volume exceeded 1500 mm³.

Statistical analysis: All of the experiments were repeated at least three times. Statistical significance was calculated using two-way ANOVA followed by Tukey's test. Survival curve was analyzed by log-rank test using GraphPad Prism 8 software.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
[1] B. A. Chabner, T. G. Roberts, Chemotherapy and the war on cancer, Nature Reviews Cancer, 5 (2005) 65-72.
[2] H. Bardania, S. Tarvirdipour, F. Dorkoosh, Liposome-targeted delivery for highly potent drugs, Artificial Cells, Nanomedicine, and Biotechnology, 45 (2017) 1478-1489.
[3] S. Senapati, A. K. Mahanta, S. Kumar, P. Maiti, Controlled drug delivery vehicles for cancer treatment and their performance, Signal Transduction and Targeted Therapy, 3 (2018) 7.
[4] F. Kratz, Albumin as a drug carrier: design of prodrugs, drug conjugates and nanoparticles, J Control Release, 132 (2008) 171-183.
[5] D. Sleep, J. Cameron, L. R. Evans, Albumin as a versatile platform for drug half-life extension, Biochim Biophys Acta, 1830 (2013) 5526-5534.
[6] F. Kratz, B. Elsadek, Clinical impact of serum proteins on drug delivery, J Control Release, 161 (2012) 429-445.
[7] A. M. Merlot, D. S. Kalinowski, D. R. Richardson, Unraveling the mysteries of serum albumin-more than just a serum protein, Front Physiol, 5 (2014) 299-299.
[8] D. D. Von Hoff, T. Ervin, F. P. Arena, E. G. Chiorean, J. Infante, M. Moore, T. Seay, S. A. Tjulandin, W. W. Ma, M. N. Saleh, M. Harris, M. Reni, S. Dowden, D. Laheru, N. Bahary, R. K. Ramanathan, J. Tabernero, M. Hidalgo, D. Goldstein, E. Van Cutsem, X. Wei, J. Iglesias, M. F. Renschler, Increased survival in pancreatic cancer with nab-paclitaxel plus gemcitabine, N Engl J Med, 369 (2013) 1691-1703.
[9] M. B. Caspersen, M. Kuhlmann, K Nicholls, M. J. Saxton, B. Andersen, K. Bunting, J. Cameron, K. A. Howard, Albumin-based drug delivery using cysteine 34 chemical conjugates—important considerations and requirements, Ther Deliv, 8 (2017) 511-519.
[10] J. G. Mehtala, C. Kulczar, M. Lavan, G. Knipp, A. Wei, Cys34-PEGylated Human Serum Albumin for Drug Binding and Delivery, Bioconjugate chemistry, 26 (2015) 941-949.
[11] A. Dag, Y. Jiang, K. J. Karim, G. Hart-Smith, W. Scarano, M. H. Stenzel, Polymer-albumin conjugate for the facilitated delivery of macromolecular platinum drugs, Macromol Rapid Commun, 36 (2015) 890-897.
[12] K. L. Heredia, D. Bontempo, T. Ly, J. T. Byers, S. Halstenberg, H. D. Maynard, In situ preparation of protein-“smart” polymer conjugates with retention of bioactivity, J Am Chem Soc, 127 (2005) 16955-16960.
[13] Z. Liu, X. Chen, Simple bioconjugate chemistry serves great clinical advances: albumin as a versatile platform for diagnosis and precision therapy, Chem Soc Rev, 45 (2016) 1432-1456.
[14] L. Pes, S. D. Koester, J. P. Magnusson, S. Chercheja, F. Medda, K. Abu Ajaj, D. Rognan, S. Daum, F. I. Nollmann, J. Garcia Fernandez, P. Perez Galan, H. K. Walter, A. Warnecke, F. Kratz, Novel auristatin E-based albumin-binding prodrugs with superior anticancer efficacy in vivo compared to the parent compound, J Control Release, 296 (2019) 81-92.
[15] D. Zhao, H. Zhang, W. Tao, W. Wei, J. Sun, Z. He, A rapid albumin-binding 5-fluorouracil prodrug with a prolonged circulation time and enhanced antitumor activity, Biomater Sci, 5 (2017) 502-510.
[16] W. Wei, C. Luo, J. Yang, B. Sun, D. Zhao, Y. Liu, Y. Wang, W. Yang, Q. Kan, J. Sun, Z. He, Precisely albumin-hitchhiking tumor cell-activated reduction/oxidation-responsive docetaxel prodrugs for the hyperselective treatment of breast cancer, J Control Release, 285 (2018) 187-199.
[17] F. E. Chamberlain, R. L. Jones, S. P. Chawla, Aldoxorubicin in soft tissue sarcomas, Future Oncol, 15 (2019) 1429-1435.
[18] E. Sachdev, D. Sachdev, M. Mita, Aldoxorubicin for the treatment of soft tissue sarcoma, Expert Opin Investig Drugs, 26 (2017) 1175-1179.
[19] S. P. Chawla, K. N. Ganjoo, S. Schuetze, Z. Papai, B. A. Van Tine, E. Choy, D. A. Liebner, M. Agulnik, S. Chawla, S. Wieland, D. J. Levitt, Phase III study of aldoxorubicin vs investigators' choice as treatment for relapsed/refractory soft tissue sarcomas, Journal of Clinical Oncology, 35 (2017) 11000-11000.
[20] S. P. Chawla, Z. Papai, G. Mukhametshina, K. Sankhala, L. Vasylyev, A. Fedenko, K. Khamly, K. Ganjoo, R. Nagarkar, S. Wieland, D. J. Levitt, First-Line Aldoxorubicin vs Doxorubicin in Metastatic or Locally Advanced Unresectable Soft-Tissue Sarcoma: A Phase 2b Randomized Clinical Trial, JAMA Oncol, 1 (2015) 1272-1280.
[21] B. Sahaf, K. Heydari, L. A. Herzenberg, L. A. Herzenberg, Lymphocyte surface thiol levels, Proc Natl Acad Sci USA, 100 (2003) 4001-4005.
[22] N. E. Sharpless, M. Flavin, The reactions of amines and amino acids with maleimides. Structure of the reaction products deduced from infrared and nuclear magnetic resonance spectroscopy, Biochemistry, 5 (1966) 2963-2971.
[23] P. Yousefpour, L. Ahn, J. Tewksbury, S. Saha, S. A. Costa, J. J. Bellucci, X. Li, A. Chilkoti, Conjugate of Doxorubicin to Albumin-Binding Peptide Outperforms Aldoxorubicin, Small, 15 (2019) e1804452.
[24] B. Gikanga, N. S. Adeniji, T. W. Patapoff, H. W. Chih, L. Yi, Cathepsin B Cleavage of vcMMAE-Based Antibody-Drug Conjugate Is Not Drug Location or Monoclonal Antibody Carrier Specific, Bioconjug Chem, 27 (2016) 1040-1049.
[25] Y. Li, C. Gu, J. Gruenhagen, P. Yehl, N. P. Chetwyn, C. D. Medley, An enzymatic deconjugation method for the analysis of small molecule active drugs on antibody-drug conjugates, MAbs, 8 (2016) 698-705.
[26] S. O. Doronina, B. E. Told, M. Y. Torgov, B. A. Mendelsohn, C. G. Cerveny, D. F. Chace, R. L. DeBlanc, R. P. Gearing, T. D. Bovee, C. B. Siegall, J. A. Francisco, A. F. Wahl, D. L. Meyer, P. D. Senter, Development of potent monoclonal antibody auristatin conjugates for cancer therapy, Nat Biotechnol, 21 (2003) 778-784.
[27] F. Monacelli, D. Storace, C. D'Arrigo, R. Sanguineti, R. Borghi, D. Pacini, A. L. Furfaro, M. A. Pronzato, P. Odetti, N. Traverso, Structural alterations of human serum albumin caused by glycative and oxidative stressors revealed by circular dichroism analysis, Int J Mol Sci, 14 (2013) 10694-10709.
[28] Y. Zhang, M. Huo, J. Zhou, S. Xie, PKSolver: An add-in program for pharmacokinetic and pharmacodynamic data analysis in Microsoft Excel, Comput Methods Programs Biomed, 99 (2010) 306-314.
[29] K. J. Hamblett, P. D. Senter, D. F. Chace, M. M. Sun, J. Lenox, C. G. Cerveny, K. M. Kissler, S. X. Bernhardt, A. K. Kopcha, R. F. Zabinski, D. L. Meyer, J. A. Francisco, Effects of drug loading on the antitumor activity of a monoclonal antibody drug conjugate, Clin Cancer Res, 10 (2004) 7063-7070.
[30] E. L. Deer, J. Gonzalez-Hernandez, J. D. Coursen, J. E. Shea, J. Ngatia, C. L. Scaife, M. A. Firpo, S. J. Mulvihill, Phenotype and genotype of pancreatic cancer cell lines, Pancreas, 39 (2010) 425-435.
[31] C. Commisso, S. M. Davidson, R. G. Soydaner-Azeloglu, S. J. Parker, J. J. Kamphorst, S. Hackett, E. Grabocka, M. Nofal, J. A. Drebin, C. B. Thompson, J. D. Rabinowitz, C. M. Metallo, M. G. Vander Heiden, D. Bar-Sagi, Macropinocytosis of protein is an amino acid supply route in Ras-transformed cells, Nature, 497 (2013) 633-637.
[32] H. Liu, M. Sun, Z. Liu, C. Kong, W. Kong, J. Ye, J. Gong, D. C. S. Huang, F. Qian, KRAS-enhanced macropinocytosis and reduced FcRn-mediated recycling sensitize pancreatic cancer to albumin-conjugated drugs, Journal of Controlled Release, 296 (2019) 40-53.
[33] X. Liu, D. Ghosh, Intracellular nanoparticle delivery by oncogenic KRAS-mediated macropinocytosis, Int J Nanomedicine, 14 (2019) 6589-6600.
[34] M. T. Larsen, M. Kuhlmann, M. L. Hvam, K. A. Howard, Albumin-based drug delivery: harnessing nature to cure disease, Mol Cell Ther, 4 (2016) 3.
[35] S. Daum, J. P. Magnusson, L. Pes, J. Garcia Fernandez, S. Chercheja, F. Medda, F. I. Nollmann, S. D. Koester, P. Perez Galan, A. Warnecke, K. Abu Ajaj, F. Kratz, Development of a Novel Imaging Agent for Determining Albumin Uptake in Solid Tumors, Nucl Med Mol Imaging, 53 (2019) 189-198.
[36] F. Kratz, R. Muller-Driver, I. Hofmann, J. Drevs, C. Unger, A novel macromolecular prodrug concept exploiting endogenous serum albumin as a drug carrier for cancer chemotherapy, J Med Chem, 43 (2000) 1253-1256.
[37] B. Elsadek, R. Graeser, N. Esser, C. Schafer-Obodozie, K. Abu Ajaj, C. Unger, A. Warnecke, T. Saleem, N. El-Melegy, H. Madkor, F. Kratz, Development of a novel prodrug of paclitaxel that is cleaved by prostate-specific antigen: an in vitro and in vivo evaluation study, Eur J Cancer, 46 (2010) 3434-3444.
[38] A. Warnecke, I. Fichtner, G. Sass, F. Kratz, Synthesis, cleavage profile, and antitumor efficacy of an albumin-binding prodrug of methotrexate that is cleaved by plasmin and cathepsin B, Arch Pharm (Weinheim), 340 (2007) 389-395.
[39] P. K. Mazur, A. Herner, F. Neff, J. T. Siveke, Current methods in mouse models of pancreatic cancer, Methods Mol Biol, 1267 (2015) 185-215.
[40] K. Temming, D. L. Meyer, R. Zabinski, E. C. F. Dijkers, K. Poelstra, G. Molema, R. J. Kok, Evaluation of RGD-Targeted Albumin Carriers for Specific Delivery of Auristatin E to Tumor Blood Vessels, Bioconjugate Chemistry, 17 (2006) 1385-1394.
[41] G. Stehle, H. Sinn, A. Wunder, H. H. Schrenk, S. Schutt, W. Maier-Borst, D. L. Heene, The loading rate determines tumor targeting properties of methotrexate-albumin conjugates in rats, Anticancer Drugs, 8 (1997) 677-685.
[42] X. Sun, J. F. Ponte, N.C. Yoder, R. Laleau, J. Coccia, L. Lanieri, Q. Qiu, R. Wu, E. Hong, M. Bogalhas, L. Wang, L. Dong, Y. Setiady, E. K. Maloney, O. Ab, X. Zhang, J. Pinkas, T. A. Keating, R. Chari, H. K. Erickson, J. M. Lambert, Effects of Drug-Antibody Ratio on Pharmacokinetics, Biodistribution, Efficacy, and Tolerability of Antibody-Maytansinoid Conjugates, Bioconjug Chem, 28 (2017) 1371-1381.
[43] J. Y. Park, M. G. Song, W. H. Kim, K. W. Kim, N. A. Lodhi, J. Y. Choi, Y. J. Kim, J. Y. Kim, H. Chung, C. Oh, Y.-S. Lee, K. W. Kang, H.-J. Im, S. H. Seok, D. S. Lee, E. E. Kim, J. M. Jeong, Versatile and Finely Tuned Albumin Nanoplatform based on Click Chemistry, Theranostics, 9 (2019) 3398-3409.
[44] M. M. Sun, K. S. Beam, C. G. Cerveny, K. J. Hamblett, R. S. Blackmore, M. Y. Torgov, F. G. Handley, N.C. Ihle, P. D. Senter, S. C. Alley, Reduction-alkylation strategies for the modification of specific monoclonal antibody disulfides, Bioconjug Chem, 16 (2005) 1282-1290.
[45] A. Beck, L. Goetsch, C. Dumontet, N. Corvaia, Strategies and challenges for the next generation of antibody-drug conjugates, Nat Rev Drug Discov, 16 (2017) 315-337.
[46] A. Sparreboom, C. D. Scripture, V. Trieu, P. J. Williams, T. De, A. Yang, B. Beals, W. D. Figg, M. Hawkins, N. Desai, Comparative Preclinical and Clinical Pharmacokinetics of a Cremophor-Free, Nanoparticle Albumin-Bound Paclitaxel (ABI-007) and Paclitaxel Formulated in Cremophor (Taxol), Clinical Cancer Research, 11 (2005) 4136.
[47] F.-F. An, X.-H. Zhang, Strategies for Preparing Albumin-based Nanoparticles for Multifunctional Bioimaging and Drug Delivery, Theranostics, 7 (2017) 3667-3689.
[48] J. E. Schnitzer, A. Sung, R. Horvat, J. Bravo, Preferential interaction of albumin-binding proteins, gp30 and gp18, with conformationally modified albumins. Presence in many cells and tissues with a possible role in catabolism, J Biol Chem, 267 (1992) 24544-24553.
[49] Y. Iwao, M. Anraku, M. Hiraike, K. Kawai, K. Nakajou, T. Kai, A. Suenaga, M. Otagiri, The structural and pharmacokinetic properties of oxidized human serum albumin, advanced oxidation protein products (AOPP), Drug Metab Pharmacokinet, 21 (2006) 140-146.
[50] R. Bito, S. Hino, A. Baba, M. Tanaka, H. Watabe, H. Kawabata, Degradation of oxidative stress-induced denatured albumin in rat liver endothelial cells, Am J Physiol Cell Physiol, 289 (2005) C531-542.
[51] S. Nie, Understanding and overcoming major barriers in cancer nanomedicine, Nanomedicine (Lond), 5 (2010) 523-528.
[52] J. A. Francisco, C. G. Cerveny, D. L. Meyer, B. J. Mixan, K. Klussman, D. F. Chace, S. X. Rejniak, K. A. Gordon, R. DeBlanc, B. E. Toki, C. L. Law, S. O. Doronina, C. B. Siegall, P. D. Senter, A. F. Wahl, cAC10-vcMMAE, an anti-CD30-monomethyl auristatin E conjugate with potent and selective antitumor activity, Blood, 102 (2003) 1458-1465.
[53] A. T. Lucas, R. Robinson, A. N. Schorzman, J. A. Piscitelli, J. F. Razo, W. C. Zamboni, Pharmacologic Considerations in the Disposition of Antibodies and Antibody-Drug Conjugates in Preclinical Models and in Patients, Antibodies (Basel), 8 (2019) 3.
[54] F. H. Igney, P. H. Krammer, Immune escape of tumors: apoptosis resistance and tumor counterattack, J Leukoc Biol, 71 (2002) 907-920.
[55] A. H. Staudacher, M. P. Brown, Antibody drug conjugates and bystander killing: is antigen-dependent internalisation required?, Br J Cancer, 117 (2017) 1736-1742.
[56] S. Eser, M. Messer, P. Eser, A. von Werder, B. Seidler, M. Bajbouj, R. Vogelmann, A. Meining, J. von Burstin, H. Algul, P. Pagel, A. E. Schnieke, I. Esposito, R. M. Schmid, G. Schneider, D. Saur, In vivo diagnosis of murine pancreatic intraepithelial neoplasia and early-stage pancreatic cancer by molecular imaging, Proc Natl Acad Sci USA, 108 (2011) 9945-9950.
[57] M. T. Schutte, R. Mulhaupt, F. Kratz, Development of Acid-Sensitive Platinum(II) Complexes With Protein-Binding Properties, Met Based Drugs, 7 (2000) 89-100.
[58] F. Kratz, DOXO-EMCH (INNO-206): the first albumin-binding prodrug of doxorubicin to enter clinical trials, Expert Opin Investig Drugs, 16 (2007) 855-866.
[59] M. Dorywalska, R. Dushin, L. Moine, S. E. Farias, D. Zhou, T. Navaratnam, V. Lui, A. Hasa-Moreno, M. G. Casas, T. T. Tran, K. Delaria, S. H. Liu, D. Foletti, C. J. O'Donnell, J. Pons, D. L. Shelton, A. Rajpal, P. Strop, Molecular Basis of Valine-Citrulline-PABC Linker Instability in Site-Specific ADCs and Its Mitigation by Linker Design, Mol Cancer Ther, 15 (2016) 958-970.

What is claimed is:

1. A composition comprising an albumin drug conjugate wherein the albumin is recombinant human albumin.

2. The composition of claim 1, wherein the drug is covalently conjugated to Cysteine 34 of the recombinant human albumin.

3. The composition of claim 1 or 2, wherein the drug and recombinant human albumin are conjugated ex vivo.

4. The composition of any of claims 1-3, wherein the albumin drug conjugate does not comprise endogenous albumin.

5. The composition of claim 4, wherein recombinant human serum albumin is at a concentration of 5 mg/mL to 15 mg/mL.

6. The composition of claim 4, wherein recombinant human serum albumin is at a concentration of 10 mg/mL.

7. The composition of any of claims 1-6, further comprising a linker positioned between the drug and the recombinant human albumin.

8. The composition of claim 7, wherein the linker is a cleavable linker.

9. The composition of claim 7 or 8, wherein the linker is conjugated to a free thiol of Cysteine 34 of albumin.

10. The composition of any of claims 1-9, wherein the linker is an enzyme sensitive linker, a pH-sensitive linker, or a reducible linker.

11. The composition of any of claims 1-10, wherein the linker is a protease sensitive linker.

12. The composition of claim 11, wherein the protease is cathepsin-B, matrix metalloproteinase, caspase-3, A disintegrin and metalloproteinase (ADAM), allekrin-related peptidase, urokinase plasminogen activator (uPA), hepsin (HPN), matripase, legumain, dipeptidyl peptidase (DPP4), or fibroblast activation protein (FAP).

13. The composition of claim 11, wherein the protease is cathepsin-B.

14. The composition of any of claim 1-12, wherein the cleavable linker is a valine-citrulline dipeptide linker.

15. The composition of any of claims 1-14, wherein the cleavable linker is a cathepsin-B sensitive valine-citrulline dipeptide linker.

16. The composition of any of claims 1-15, wherein the albumin and drug-linker conjugate are conjugated at a molar ratio of 1:1 to 1:5.

17. The composition of any of claims 1-16, wherein the albumin and drug-linker conjugate are conjugated at a molar ratio of 1:3.

18. The composition of any of claims 1-17, wherein the albumin drug conjugate further comprises a spacer.

19. The composition of claim 18, wherein the spacer is a a p-aminobenzyl carbamate (PABC) spacer, PEG spacers, or carbamoyl sulfamide linker.

20. The composition of claim 18, wherein the spacer is a p-aminobenzyl carbamate (PABC) spacer.

21. The composition of any of claims 18-20, wherein the spacer is located between the drug the linker.

22. The composition of any of claims 1-21, wherein the molar ratio of drug to albumin is 1:1 to 3:1.

23. The composition of any of claims 1-22, wherein the molar ratio of drug to albumin is 1:1.

24. The composition of any of claims 1-23, wherein the drug is an anti-cancer agent.

25. The composition of any of claims 1-24, wherein the drug is chemotherapy, radiotherapy, gene therapy, hormonal therapy, anti-angiogenic therapy or immunotherapy.

26. The composition of claim 24, wherein the anti-cancer agent is a SHP inhibitor, a SOS inhibitor, a maytansinoid, an auristatin, calicheamicin, an anthracycline, a taxane, a MEK inhibitor, a poly (adenosine diphosphate ribose) polymerase (PARP) inhibitor, a RAF inhibitor, or a KRAS G12C inhibitor, platinum-based compound, topoisomerase I inhibitor or anthracycline.

27. The composition of claim 24, wherein the anti-cancer agent is a chemotherapeutic agent.

28. The composition of any of claims 1-27, wherein the drug is monomethyl auristatin E (MMAE) or gemcitabine.

29. The composition of claim 27, wherein the chemotherapeutic agent is anthracycline, camptothecin, paclitaxel, auristatin, or docetaxel.

30. The composition of any of claims 1-29, further defined as a pharmaceutical composition.

31. The pharmaceutical composition of claim 30 for use in the treatment of cancer in a subject.

32. The use of claim 31, wherein the cancer is a RAS mutant cancer.

33. The use of claim 31 or 32, wherein the cancer is pancreatic cancer, lung cancer, or colorectal cancer.

34. The use of any of claims 31-33, wherein the subject is human.

35. A method of delivering a drug into a tumor cell comprising administering an effective amount of the pharmaceutical composition of claim 30 to said cell.

36. A method of treating cancer in a subject comprising administering an effective amount of the pharmaceutical composition of claim 30 to said subject.

37. The method of claim 36, wherein the cancer is a RAS mutant cancer.

38. The method of claim 37, wherein the RAS mutant cancer is pancreatic cancer, colorectal cancer, or lung cancer.

39. The method of any of claims 36-38, wherein the cancer is pancreatic cancer.

40. The method of any of claims 36-39, wherein the subject is a human.

41. The method of any of claims 36-40, wherein the albumin drug conjugate is administered orally, topically, intravenously, intraperitoneally, intramuscularly, endoscopically, percutaneously, subcutaneously, regionally, or by direct injection.

42. The method of any of claims 36-41, wherein the albumin drug conjugate is administered intravenously.

43. The method of any of claims 36-42, further comprising administering at least a second therapeutic agent.

44. The method of claim 43, wherein the at least a second therapeutic agent is an anti-cancer agent.

45. The method of claim 43 or 44, wherein the at least a second therapeutic is chemotherapy, radiotherapy, gene therapy, surgery, hormonal therapy, anti-angiogenic therapy or immunotherapy.

46. The method of any of claims 36-44, wherein the albumin drug conjugate has improved half-life, anti-tumor efficacy, and/or is delivered at a higher dose to a tumor as compared to an albumin drug conjugated in vivo.

47. The method of claim 45, wherein the at least a second therapeutic is immunotherapy.

48. The method of claim 45, wherein the immunotherapy is a cytokine or STING agonist.

49. The method of claim 48, wherein the cytokine is IL-2 or IL-12.

50. The method of claim 47, wherein the immunotherapy is an immune checkpoint inhibitor.

51. The method of claim 50, wherein the immune checkpoint inhibitor is an inhibitor of an inhibitor of CTLA-4, PD-1, PD-L1, PD-L2, LAG-3, BTLA, B7H3, B7H4, TIM3, KIR, or A2aR, TIGIT, or VISTA.

52. The method of claim 50, wherein the immune checkpoint inhibitor is an anti-PD1 antibody.

53. The method of claim 52, wherein the anti-PD1 antibody is nivolumab, pembrolizumab, pidillizumab, KEYTRUDA®, AMP-514, REGN2810, CT-011, BMS 936559, MPDL328OA or AMP-224.

54. The method of claim 50, wherein the at least one immune checkpoint inhibitor is an anti-CTLA-4 antibody.

55. The method of claim 54, wherein the anti-CTLA-4 antibody is tremelimumab, YERVOY®, or ipilimumab.

56. A method for producing an albumin drug conjugate comprising covalently conjugating a drug to Cysteine 34 of albumin, wherein the conjugation is performed ex vivo.

57. The method of claim 56, wherein the albumin is recombinant human serum albumin.

58. The method of claim 57, wherein recombinant human serum albumin is at a concentration of 5 mg/mL to 15 mg/mL.

59. The method of claim 57, wherein recombinant human serum albumin is at a concentration of 10 mg/mL.

60. The method of any of claims 56-59, wherein the drug is conjugated to a linker prior to conjugating to albumin.

61. The method of claim 60, wherein the linker is a cleavable linker.

62. The method of claim 60 or 61, wherein the linker conjugates to a free thiol of Cysteine 34 of albumin.

63. The method of any of claims 56-62, wherein the linker is an enzyme sensitive linker, a pH-sensitive linker, or a reducible linker.

64. The method of any of claims 56-63, wherein the linker is a protease sensitive linker.

65. The method of claim 63, wherein the protease is cathepsin-B, matrix metalloproteinase, caspase-3, A disintegrin and metalloproteinase (ADAM), allekrin-related peptidase, urokinase plasminogen activator (uPA), hepsin (HPN), matripase, legumain, dipeptidyl peptidase (DPP4), or fibroblast activation protein (FAP).

66. The method of claim 63, wherein the protease is cathepsin-B.

67. The method of any of claim 56-65, wherein the cleavable linker is a valine-citrulline dipeptide linker.

68. The method of any of claims 56-67, wherein the cleavable linker is a cathepsin-B sensitive valine-citrulline dipeptide linker.

69. The method of any of claims 56-68, wherein the albumin is added to a drug-linker conjugate at a molar ratio of 1:1 to 1:5.

70. The method of any of claims 56-69, wherein the albumin is added to a drug-linker conjugate at a molar ratio of 1:3.

71. The method of claim 70, wherein excess drug-linker conjugate is removed by a desalting column or flow filtration.

72. The method of any of claims 56-71, wherein the albumin is dissolved in phosphate buffered saline.

73. The method of any of claims 56-72, wherein the drug-linker conjugate is dissolved in acetonitrile.

74. The method of any of claims 56-73, wherein the albumin drug conjugate does not comprise endogenous albumin.

75. The method of any of claims 56-74, wherein the albumin drug conjugate further comprises a spacer.

76. The method of claim 75, wherein the spacer is a a p-aminobenzyl carbamate (PABC) spacer, PEG spacers, or carbamoyl sulfamide linker.

77. The method of claim 75, wherein the spacer is a p-aminobenzyl carbamate (PABC) spacer.

78. The method of claim 75 or 77, wherein the spacer is located between the drug the the linker.

79. The method of any of claims 56-78, wherein the molar ratio of drug to albumin is 1:1 to 3:1.

80. The method of any of claims 56-79, wherein the molar ratio of drug to albumin is 1:1.

81. The method of any of claims 56-80, further comprising reducing albumin to expose reactive thiols prior to conjugation.

82. The method of claim 81, wherein reducing comprises the addition of tris(2-carboxyethyl) phosphine hydrochloride (TCEP).

83. The method of any of claims 56-82, wherein the drug is an anti-cancer agent.

84. The method of any of claims 56-83, wherein the drug is chemotherapy, radiotherapy, gene therapy, hormonal therapy, anti-angiogenic therapy or immunotherapy.

85. The method of claim 83, wherein the anti-cancer agent is a SHP inhibitor, a SOS inhibitor, a maytansinoid, an auristatin, calicheamicin, an anthracycline, a taxane, a MEK inhibitor, a poly (adenosine diphosphate ribose) polymerase (PARP) inhibitor, a RAF inhibitor, or a KRAS G12C inhibitor, platinum-based compound, topoisomerase I inhibitor or anthracycline.

86. The method of claim 83, wherein the anti-cancer agent is a chemotherapeutic agent.

87. The method of any of claims 56-86, wherein the drug is monomethyl auristatin E (MMAE) or gemcitabine.

88. The method of claim 86, wherein the chemotherapeutic agent is anthracycline, camptothecin, paclitaxel, auristatin, or docetaxel.
